(19) United States
(12) Patent Application Publication
(10) Pub. No.: US 2026/0199414 A1
(43) Pub. Date: Jul. 16, 2026

(54) RECOMBINANT VACCINIA VIRUS ENCODING ONE OR MORE NATURAL KILLER CELL AND T LYMPHOCYTE INHIBITORS

(71) Applicant: TURNSTONE BIOLOGICS CORP., La Jolla, CA (US)

(72) Inventors: Sebastien Delpeut, Ottawa (CA); David Stojdl, Ottawa (CA)

(21) Appl. No.: 19/139,263

(22) PCT Filed: Dec. 15, 2023
(86) PCT No.: PCT/US2023/084453

§ 371 Date: Jun. 13, 2025

Related U.S. Application Data
(60) Provisional application No. 63/433,414, filed on Dec. 16, 2022.
(60) Provisional application No. 63/433,743, filed on Dec. 19, 2022.

(51) Int. Cl. A61K 35/76; A61K 35/17; A61K 40/11; A61K 40/31; A61K 45/06; A61P 35/00; C07K 14/54; C07K 16/28; C12N 7/00; C12N 15/11

(52) U.S. Cl. CPC A61K 35/76; A61K 35/17; A61K 40/11; A61K 40/31; A61K 45/06; A61P 35/00; C07K 14/5434; C07K 16/2818; C12N 7/00; C12N 15/11; C12N 2710/24121; C12N 2710/24132; C12N 2830/34

(57) ABSTRACT

Various embodiments of the invention provide recombinant oncolytic viruses engineered to express human cytomegalovirus (HCMV) glycoprotein UL40 and/or Kaposi's sarcoma associated herpesvirus (KSHV) K5 protein, and methods and uses of the same for treating cancer. Also provided are combination therapies involving a T lymphocyte infiltrating (TIL) cell therapy and a provided recombinant oncolytic virus for treating a cancer, including solid tumors.

### CROSS-REFERENCE TO RELATED APPLICATIONS

This application claims priority to U.S. Provisional Patent Application No. 63/433,414, filed Dec. 16, 2022, entitled “RECOMBINANT VACCINIA VIRUS ENCODING ONE OR MORE NATURAL KILLER CELL AND T LYMPHOCYTE INHIBITORS,” and to U.S. Provisional Patent Application No. 63/433,743, filed Dec. 19, 2022, entitled “RECOMBINANT VACCINIA VIRUS ENCODING ONE OR MORE NATURAL KILLER CELL AND T LYMPHOCYTE INHIBITORS,” which are herein incorporated by reference in their entirety for all purposes.

### INCORPORATION BY REFERENCE OF SEQUENCE LISTING

The present application is being filed along with a Sequence Listing in electronic format. The Sequence Listing is provided as a file entitled 220612001440SeqList.xml, created Dec. 15, 2023, which is 724,548 bytes in size. The information in the electronic format of the Sequence Listing is herein incorporated by reference in its entirety.

### FIELD OF THE INVENTION

Embodiments of the invention relate to recombinant oncolytic viruses engineered to express human cytomegalovirus (HCMV) glycoproteins UL40 and/or Kaposi's sarcoma associated herpesvirus (KSHV) protein. More specifically embodiments of the invention relate to recombinant oncolytic viruses engineered to express human cytomegalovirus (HCMV) glycoprotein UL40 and/or Kaposi's sarcoma associated herpesvirus (KSHV) K5 protein, and methods and uses of the same for treating cancer. Embodiments also relate to combination therapies involving a T lymphocyte infiltrating (TIL) cell therapy and a provided recombinant oncolytic virus expressing one or both of HCMV glycoprotein UL40 and/or KSHV K5 protein for treating cancer, including solid tumors.

### BACKGROUND

Solid tumors remain a leading cancer affecting millions of new patients each year. Although various strategies are available for treating cancers, including solid tumors, there are still obstacles with existing approaches. Targeted oncology has demonstrated some progress but with few cures. For instance, use of oncolytic viruses show promise but have limitations. One such limitation is immune clearance of the oncolytic viruses, where the patient's immune system clears the virus before the oncolytic effects are realized. Thus, there is a need for improved oncolytic viruses and methods of using, including for treating solid tumors. Provided are embodiments that meet such needs.

### SUMMARY OF THE INVENTION

Various embodiments of the invention provide a nucleic acid comprising a recombinant Orthopoxvirus genome and one or more transgenes comprising (a) a nucleotide sequence encoding UL40 and/or (b) a nucleotide sequence encoding K5.

In some embodiments, the recombinant Orthopoxvirus genome is derived from a Vaccinia virus genome. In some embodiments, the Vaccinia virus genome is derived from a genome of a Copenhagen strain of Vaccinia virus.

In some of any embodiment the Orthopoxvirus genome comprises: (a) deletions in one or more of the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R; (b) deletions in one or more of the following genes in the 3′ inverted terminal repeat (ITR): B21R, B22R, B23R, B24R, B25R, B26R, B27R, B28R, and B29R; and/or (c) deletion in the B8R gene. In some of any embodiments, the Orthopoxvirus genome comprises: (a) deletions in the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R and B20R; (b) deletions in the following genes in the 3′ inverted terminal repeat (ITR): B21R, B22R, B23R, B24R, B25R, B26R, B27R, B28R and B29R; and (c) deletion in the B8R gene. In some of any embodiments, the deletions in the C2L, F3L, B14R, and B29R vaccinia genes are partial deletions.

In some of any embodiments, the Orthopoxvirus genome has at least 95% sequence identity to SEQ ID NO 15 In some of any embodiments, the Orthopoxvirus genome has the sequence set forth in SEQ ID NO 15.

In some of any embodiments, the Orthopoxvirus genome comprises a nucleotide sequence encoding UL40.

In some of any embodiments, the Orthopoxvirus genome comprises a nucleotide sequence encoding K5.

In some of any embodiments, the Orthopoxvirus genome comprises (a) a nucleotide sequence encoding UL40 and (b) a nucleotide sequence encoding K5.

In some of any embodiments, the nucleotide sequence encoding UL40 encodes an amino acid sequence with at least 95% sequence identity to SEQ ID NO 3. In some of any embodiments, the nucleotide sequence encoding UL40 encodes the amino acid sequence set forth in SEQ ID NO 3.

In some of any embodiments, the nucleotide sequence encoding UL40 comprises a sequence with at least 95% sequence identity to SEQ ID NO 1. In some of any embodiments, the nucleotide sequence encoding UL40 comprises the sequence set forth in SEQ ID NO 1.

In some of any embodiments, the nucleotide sequence encoding UL40 is operably linked to a vaccinia virus early/late promoter.

In some of any embodiments, the nucleotide sequence encoding K5 encodes an amino acid sequence with at least 95% sequence identity to SEQ ID NO 6. In some of any embodiments, the nucleotide sequence encoding K5 encodes the amino acid sequence set forth in SEQ ID NO 6.

In some of any embodiments, the nucleotide sequence encoding K5 comprises a sequence with at least 95% sequence identity to SEQ ID NO 2. In some of any embodiments, the nucleotide sequence encoding K5 comprises the sequence set forth in SEQ ID NO 2.

In some of any embodiments, the nucleotide sequence encoding K5 is operably linked to a vaccinia virus early/late promoter.

In some of any embodiments, the one or more transgenes comprises at least one further transgene comprising a nucleotide sequence encoding an immunomodulatory protein selected from the group consisting of a checkpoint inhibitor, an interleukin, a cytokine and an NK cell and/or T cell inhibitor.

In some embodiments, the immunomodulatory protein is FMS-like tyrosine kinase 3 ligand (FLT3L), an antibody that specifically binds CTLA-4, or an Interleukin 12 (IL-12) polypeptide, optionally wherein the IL-12 polypeptide is a membrane-bound IL-12.

In some of any embodiments, the nucleotide sequence encoding the at least one further transgene is operably linked to a vaccinia virus early/late promoter.

In some of any embodiments, the vaccinia virus early/late promoter is selected from one or more of H5R, P7.5, and E3L, or is selected from SEQ ID NO: 4, SEQ ID NO: 5, and SEQ ID NO:14.

In some of any embodiments, the one or more transgenes is inserted into locus 5p of the recombinant Orthopoxvirus genome between the genes C2L and F3L.

In some of any embodiments, the one or more transgenes is inserted into locus 3p of the recombinant Orthopoxvirus genome between the genes B14R and B29R.

In some of any embodiments, the one or more transgenes are in the same orientation as endogenous Orthopoxvirus genes within the recombinant Orthopoxvirus genome.

In some of any embodiments, expression of UL40 increases HLA-E surface expression in a host cell infected by a recombinant Orthopoxvirus comprising the nucleic acid. In some of any embodiments, expression of K5 decreases HLA-ABC surface expression on a host cell infected by a recombinant Orthopoxvirus comprising the nucleic acid. In some of any embodiments, the expression of K5 decreases ICAM-1 surface expression on a host cell infected by a recombinant Orthopoxvirus comprising the nucleic acid.

In some of any embodiments, host cells infected by a recombinant Orthopoxvirus comprising the nucleic acid are killed by lymphocytes at a reduced rate in comparison to cells infected with a reference recombinant Orthopoxvirus that has a similar genome but does not comprise a nucleotide sequence encoding either UL40 or K5.

In some of any embodiments, host cells infected by a recombinant Orthopoxvirus comprising the nucleic acid are killed by NK cells at a reduced rate in comparison to cells infected with a recombinant Orthopoxvirus that has a similar genome but does not comprise a nucleotide sequence encoding either UL40 or K5.

In some of any embodiments, host cells infected by a recombinant Orthopoxvirus comprising the nucleic acid are killed by T cells at a reduced rate in comparison to cells infected with a recombinant Orthopoxvirus that has a similar genome but does not comprise a nucleotide sequence encoding either UL40 or K5.

Provided herein is a recombinant Orthopoxvirus encoded by the nucleic acid of some of any embodiments. Provided herein is a pharmaceutical composition comprising the recombinant Orthopoxvirus of some embodiments and a physiologically acceptable carrier.

Provided herein is a method of treating cancer comprising administering the recombinant Orthopoxvirus of some embodiments to a subject Provided herein is a method of treating cancer comprising administering the pharmaceutical composition of some embodiments to a subject In some of any embodiments, the subject is human.

In some of any embodiments, the method further comprising administering a second therapeutic agent for the treatment of cancer. In some embodiments, the second therapeutic agent is administered before, concurrently with or after administering the pharmaceutical composition or recombinant Orthopoxvirus.

In various embodiments the second therapeutic agent may correspond to a cancer therapy and/or agents in used various cancer therapies. For example, in some of any embodiments, the second therapeutic agent is an autologous tumor infiltrating lymphocyte (TIL) therapy. In some embodiments, the pharmaceutical composition or recombinant Orthopoxvirus is administered to the subject prior to harvesting the TILs from a tumor from the subject for producing the autologous TIL therapy.

In some of any embodiments, the second therapeutic agent is CAR T cell therapy.

In some of any embodiments, the second therapeutic agent is a checkpoint blockade immunotherapy. In these and related embodiments, the checkpoint blockade immunotherapy is a PD1 and/or a PD-L1 inhibitor.

In some of any embodiments, the second therapeutic agent is an immunomodulatory agent. In these and related embodiments, the immunomodulatory agent may correspond to one or more of a CD47 inhibitor or NKG2A inhibitor as well as their analogues.

### BRIEF DESCRIPTION OF THE DRAWINGS

FIG. 1A-1C show uninfected cells and cells infected with parental SKV, SKV-K5, SKV-UL40, or SKV-UL40_K5 with HLA-E quantifying surface expression of HLA-E (FIG. 1A), ICAM-1 (FIG. 1B), and HLA-ABC (FIG. 1C).

FIG. 2A illustrates the ratio of cells obtained by an isolation of NK cells. FIG. 2B depicts an exemplary killing assay done with the isolated NK cells where the NK cells were incubated with HeLa cells that were uninfected or infected with parental SKV, SKV-K5, SKV-UL40, or SKV-UL40_K5 and NK cell mediated killing of the HeLa cells was measured.

FIG. 3A and FIG. 3B illustrate an exemplary experiment where MeWo cells were infected with parental SKV, SKV-K5, SKV-UL40, or SKV-UL40_K5 and incubated both with and without IFNγ before surface expression of HLA-ABC was measured (FIG. 3A), along with surface expression of HLA-E (FIG. 3B). Infected cells were then incubated with T cells to quantify T cell mediated killing of infected cells as shown in FIG. 3C.

### DETAILED DESCRIPTION OF THE INVENTION

Various embodiments of the invention provide recombinant oncolytic viruses and methods and uses involving such recombinant oncolytic viruses. In some embodiments, the genome of the recombinant oncolytic virus contains one or more transgenes. In some embodiments, recombinant oncolytic virus is encoded by a nucleic acid encoding a recombinant Orthopoxvirus genome containing the one or more transgenes. In particular embodiments, the one or more transgenes includes human cytomegalovirus (HCMV) glycoprotein UL40 and/or Kaposi's sarcoma associated herpesvirus (KSHV) K5 protein. In some embodiments the one or more transgenes are able to reduce or inhibit NK and/or T cell mediated killing of recombinant oncolytic virus infected cells. Also provided herein are methods of utilizing the recombinant oncolytic virus for treatment of cancer. Also provided are methods of utilizing the recombinant oncolytic virus in conjunction with T cell therapy, such as T lymphocyte infiltrating (TIL) cell therapy or a chimeric antigen receptor (CAR) T cell therapy.

Numerous poxvirus-based oncolytic platforms have entered clinical trials over the past decade including Orthopox viruses such as Vaccinia virus (VACV) (Chaurasiya et al. (2018) Biomed. 9:419), however, the main challenge of oncolytic virotherapy remains unsolved with premature viral clearance mediated by both innate (NK cell mediated) and adaptive (T cell mediated) antiviral immune responses.

Although oncolytic viruses provide direct cancer lysis to stimulate systemic immune response, phenotyping of CD8⁺ TILs indicates that tumor-infiltrating lymphocytes can not only be specific for tumor antigen but also recognize viral epitopes (Simoni et al. (2018) Nature. 557:575-579). In addition, immunodominant VACV-specific cytotoxic T lymphocyte (CTL) responses reduce the effectiveness of poxvirus infection in recombinant vaccination strategies (Smith et al. (2005) J Immunol. 175:8431-8437 and Harrington et al. (2002) J Virol. 76:3329-3337) which is partially attributed to the presence of long-lived VACV-specific T cell responses resulting from the worldwide immunization campaign against smallpox (Demkowic et al. (1993) J Virol. 67:1538-1544 and Demkowicz et al. (1996) J Virol. 70:2627-2631). MHC-I molecules such as HLA-A, B, or C can display proteins from an Orthopox oncolytic virus, triggering a T cell response. Several highly conserved HLA-A*0201-restricted CD8+ epitopes encoded by VACV have been identified (Terajima et al. (2002) Virus Res. 84:67-77, Ennis et al. (2002) J Infect Dis. 185: 1657-1659, and Frey et al. (2003) Jama. 289: 3295-3299). Recombinant modified vaccinia Ankara (MVA) encoding well-defined HLA-A*0201-restricted melanoma tumor antigen (Ag) epitopes generated significantly higher virus-specific CTL responses in comparison to epitope-specific CTL responses in melanoma patients (Smith et al. (2005) J Immunol. 175: 8431-8437). Croft et al. actually demonstrated that more than 80% of VACV peptides presented by MHC-I on infected mouse cells were immunogenic across a population of mice (Croft et al. (2019) Proc National Acad Sci. 116: 3112-3117). VACV-specific CD8+ T cells infiltrate melanoma lesions during acute infection and remain functional which could promote viral clearance (Erkes et al. (2017) J Immunol. 198: 2979-2988).

Vaccinia virus infection provokes increased susceptibility to NK lysis (Baraz et al. (1999) Bone Marrow Transpl. 24: 179-189, Brutkiewicz (1992) Nat Immun. 1(4):203-214, and Chisholm (2006) J Virol. 80:2225-2233) by eliciting NK activation, proliferation, and accumulation at the site of infection (Bukowski et al. (1983) 131(3):1531-1538, Daniels et al. (2001) J Exp Medicine. 194:29-44, Dokun et al. (2001) Nat Immunol. 2:951-956, and Natuk et al. (1987) J Immunol. 138(3):877-883). NK cells express activating and inhibitory receptors that regulate NK cell function (Ryan et al. (2001) Immunol Rev. 181:126-137, and Lanier (2005) Annu Rev Immunol. 23:225-274). Signals transduced by inhibitory receptors maintain NK cells in a resting state while the loss of inhibitory signals, such as the downregulation of HLA-ABC, or upregulation of ligands for activating receptors on target cells induce NK cell activation (Lanier (2005) Annu Rev Immunol. 23:225-274).

NKG2A/CD94 is an inhibitory surface receptor expressed by NK cells (Brooks et al. (1997) J Exp Medicine. 185:795-800) and a subset of CD8⁺ T cells (Llano et al. (1998) Proc National Acad Sci. 95:5199-5204). NKG2A ligand (HLA-E) usually presents peptides derived from the leader sequence of other HLA class I molecules to the NKG2A/CD94 heterodimer which inhibits NK cell-mediated lysis (Braud et al. (1998) Nature 391:795-799, Braud et al. (1997) Eur J Immunol 27:1164-1169, and Kaiser et al. (2005) J Immunol. 173:2878-2884). Thereby, HLA-E surface expression on melanoma cells decreases their susceptibility to cytotoxicity from NKG2A⁺/CD8⁺ T cells and NK cells (Braud et al. (2003) Trend Immunol. 24:162-164, and Derré et al. (2006) Cancer Immunol Res. 7:1293-1306).

NKG2D is an activating receptor that is expressed on the surface of NK, T cell, and macrophage cell lineages. NKG2D ligands are poorly expressed on normal cells but are upregulated on damaged or infected cells or on cancer cells (Zingoni et al. (2018) Front Immunol. 9:476 and Hall et al. (2019) J Immunother Cancer. 7:263). Interestingly, the engagement of NKG2D in CD8+ T cells results in enhanced TCR activation and function (Bauer et al. (1999) Science. 285:727-729, Houchins et al. (1991) J Exp. Medicine. 173:1017-1020, and Jamieson et al. (2002) Immunity. 17:19-29) which facilitates the recognition and destruction of target cells (Bauer et al. (1999) Science. 285:727-729). Thereby, NKG2D receptor signaling was shown to enhance cytolytic activity of virus specific CD8⁺ T cells (Walsh et al. (2008) J Virol. 82:3031-3044).

Interestingly, the leukocyte integrin LFA-1 is a primary adhesion molecule found on both CD8⁺ T and NK cells which binds to its ligand ICAM-1 expressed on the target cell. ICAM-1/LFA-1 interaction is a common requirement for NK and T cell-mediated cytotoxicity notably by polarizing their cytotoxic machinery towards the target cell (Urlaub et al. (2017) J Immunol. 198:1944-1951, Matumoto (1998) J Immunol. 160(12):5781-5789, Kooyk et al. (1997) Biochem Soc T. 25:515-520, Ding et al. (1999) J Immunol. 163(9):5029-5038, and Dustin et al. (1989) Cold Spring Harb Symp. 54:753-765). In addition, IFNγ is released in large amounts by NK cells and activated CD8⁺ T cells (Esteso et al. (2017) J Biol Chem 292:20472-20480, Villegas-Mendez et al. (2011) J Immunol. 187:2885-2897, and Arany et al. (1998) Arch Dermatol Res. 290:331-334) which in turn markedly increases CTL-mediated cytotoxicity by upregulating TAP and MHC-I expression on target cells (Saunders et al. (1994) J Resp Cell Mol 11:147-152, Seliger et al. (1997) Scand J Immunol. 46:625-632, and Ma et al. (1997) J Biol Chem. 272:16585-16590)

Many DNA viruses encode proteins that can overcome innate and adaptive antiviral immunity. (Iannello et al. (2006) J Leukocyte Biol. 79:16-35). To note, both human cytomegalovirus (HCMV) and Kaposi's sarcoma-associated herpesvirus (KSHV) dedicate a large portion of their genomes for modulation of host response to infection (Patro (2019) Front Immunol. 10:1155, and Lee et al. (2010) Future Microbiol. 5:1349-1365). However, whether these systems would work in other virus systems is not known, particularly given differences among viruses and factors that may impact immune evasion.

The signal peptide of HCMV UL40 contains an HLA-E ligand (Prod'homme et al. (2012) J Immunol. 188:2794-2804, and Ulbrecht et al. (2000) J Immunol. 164:5019-5022) which promotes cell surface expression of HLA-E (Tomasec et al. (2000) Science. 287:1031-1033). Binding of HLA-E/UL40 peptide complexes to the inhibitory NK cell receptor NKG2A/CD94 promotes efficient protection against lysis mediated by NKG2A/CD94 NK cells (Ulbrecht et al. (2000) J Immunol. 164:5019-5022, Tomasec et al. (2000) Science. 287:1031-1033, Wang et al. (2002) Proc National Acad Sci. 99:7570-7575, Borrego et al. (1998) J Exp. Medicine. 187:813-818, Braud et al. (1998) Nature 391:795-799, and Lee et al. (1998) Proc National Acad Sci. 95:5199-5204).

KSHV K5 is a multifunctional protein that plays a key role in evading both innate (NK cell) and adaptive (T cell) antiviral immune responses. The ubiquitin E3 ligase K5 protein downregulates cell surface expression of MHC-I molecules (HLA-A and B) and NKG2D ligands on target cells protecting them against CTL and NK cell cytotoxicity, respectively. (Li et al. (2007) J Virol. 81:2117-2127, Coscoy et al. (2000) Proc National Acad Sci. 97:8051-8056, and Thomas et al. (2008) Proc National Acad Sci. 105:1656-1661). Thereby, K5 dramatically reduces ICAM-1 surface expression on target cells which impairs T cell activation (Coscoy et al. (2001) J Clin Invest. 107:1599-1606) and drastically inhibits NK cell-mediated cytotoxicity (Ishido et al. (2000) Immunity. 13:365-374). Interestingly, K5 specifically promotes endocytosis of IFNγ receptor 1, thereby, inhibiting IFNγ action (Li et al. (2007) J Virol. 81:2117-2127)(Urlaub et al. (2017) J Immunol. 198:1944-1951, Matumoto (1998) J Immunol. 160(12):5781-5789, and Kooyk et al. (1997) Biochem Soc T. 25:515-520) (Ding et al. (1999) J Immunol. 163(9):5029-5038, Dustin et al. (1989) Cold Spring Harb Symp. 54:753-765, and Esteso et al. (2017) J Biol Chem 292:20472-20480).

The provided embodiments are based on findings that engineering the HCMV glycoprotein UL40 and/or KSHV K5 protein into the genome of a recombinant Orthopoxvirus, such as VACV, results in improved activity to reduce or inhibit innate and/or adaptive immune responses against the virus. In some embodiments, provided herein is a recombinant nucleic acid which encodes a recombinant Orthopoxvirus genome with one or more transgenes that include (a) a nucleotide sequence encoding HCMV glycoprotein UL40 and/or (b) a nucleotide sequence encoding KSHV K5 protein. In some embodiments, the nucleic acid is used to generate a recombinant Orthopoxvirus. In some embodiments, UL40 and/or K5 expression reduces or inhibits NK and/or T-cell mediated killing of Orthopoxvirus infected cells. In some embodiments, the expressed UL40 and/or K5 are retained within the cell interior which restricts their protective function to the virus-infected cells. In some embodiments, evasion from lysis induced by NK and CD8⁺ T cells promote viral persistence within the infected tumor, thereby maximizing the expression of other immunomodulatory transgenes (e.g., cytokines, chemokines, TME modifiers, bispecific T cell engagers) which also may be encoded in the recombinant nucleotide.

In provided aspects, results herein show that UL40 expressed from the recombinant nucleic acid significantly decreases T cell mediated killing of virus infected cells which is surprising as UL40 had not previously been shown to decrease T cell mediated killing. In some embodiments, surface HLA-E expression is increased on target melanoma cells used for T cell cytotoxicity-mediated killing assay when UL40 is expressed from a nucleic acid or from an Orthopoxvirus. In some embodiments, UL40 expressing Orthopoxvirus can promote viral persistence within the tumor by protecting the Orthopoxvirus infected cancer cell from tumor specific NKG2A/CD94 CD8⁺ TIL.

An explanation of why the results for UL40 and/or K5 genes were surprising will now be provided. Large-DNA viruses encode many proteins that are dedicated to host immune evasion Deletion of immunomodulatory genes is a novel genetic approach to oncolytic vaccinia virus (VACV) development, which attenuates viral replication in healthy cells but not in cancer cells. Some of VACV immunomodulatory proteins are secreted from the infected cells and act as a soluble decoy to neutralize complement factors, interferons, cytokines, and chemokines. Other VACV proteins suppress innate immune response by inhibiting signalling pathways that lead to apoptosis and production of interferon, pro-inflammatory cytokines, and chemokines

Although vaccinia virus infection can provoke increased susceptibility to lysis mediated by both NK and T cells, VACV immunomodulatory protein(s) that might contribute to the evasion of NK and T cell immunity (if any) remain(s) to be discovered. On the other hand, herpesviruses, such as HCMV and KSHV, encode numerous immunomodulatory proteins known to interfere in each step of the antigen presentation pathway and activation of NK cells. In particular, HCMV UL40 promotes efficient protection against killing mediated by NK cells while the multifunctional protein KSHV K5 can inhibit cytotoxicity-induced by NK and T cells. In the context of viral infection, HCMV and KSHV do not rely solely on UL40 and K5 but act in synergy with a myriad of other viral immunomodulatory proteins to evade both NK and T cell immunity.

Embodiments of the SKV backbone described herein are derived from the Copenhagen strain of VACV by deleting numerous immunomodulatory genes at the 5p and 3p locus as well as the B8R gene. In the studies described in the examples, the superior level of protection against NK and T-cell-mediated lysis provided by solely UL40 and/or K5 in SKV infected cells was unforeseen. Indeed, one might have expected the need to express additional immunomodulatory proteins (e.g., NK and T cell inhibitors) to reach such high level of defense against NK and T cell immunity as was observed solely for UL40 and/or K5 in SKV infected cells.

In some embodiments, the findings herein support a combination therapy of recombinant Orthopoxvirus expressing UL40(+/−K5) with an adoptive T cell therapy. In some embodiments, the T cell therapy may be an autologous bulk and/or selected tumor-infiltrating lymphocytes. In some embodiments, recombinant Orthopoxvirus can be administrated before, concurrently with or after treatment with a tumor infiltrating lymphocyte therapy.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, “optional” or “optionally” means that the subsequently described event or circumstance does or does not occur, and that the description includes instances where said event or circumstance occurs and instances where it does not. For example, an optionally substituted group means that the group is unsubstituted or is substituted.

The term “autologous” as used herein means a cell or tissue that is removed from the same organism to which it is later infused or adoptively transferred.

As used herein, a composition refers to any mixture of two or more products, substances, or compounds, including cells. It may be a solution, a suspension, liquid, powder, a paste, aqueous, non-aqueous or any combination thereof.

The term “pharmaceutical composition” refers to a composition suitable for pharmaceutical use in a mammalian subject, often a human. A pharmaceutical composition typically comprises an effective amount of an active agent (e.g., cells, such as expanded in accord with the provided methods) and a carrier, excipient, or diluent. The carrier, excipient, or diluent is typically a pharmaceutically acceptable carrier, excipient or diluent, respectively.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and are compatible with other ingredients of the formulation. The pharmaceutically acceptable carrier is appropriate for the formulation employed.

As used herein, a “subject” is a mammal, such as a human or other animal, and typically is human. The subject can be male or female and can be any suitable age, including infant, juvenile, adolescent, adult, and geriatric subjects.

The terms “effective amount” or “therapeutically effective amount” refer to a quantity and/or concentration of a therapeutic composition, such as containing cells, e.g. expanded in accord with the provide methods, that when administered to a patient yields any manner in which the symptoms of a condition, disorder or disease or other indication, are ameliorated or otherwise beneficially altered. An effective amount for treating a disease or disorder may be an amount that relieves, lessens, or alleviates at least one symptom or biological response or effect associated with the disease or disorder, prevents progression of the disease or disorder, or improves physical functioning of the patient. In particular aspects, there is a statistically significant inhibition of disease progression as, for example, by ameliorating or eliminating symptoms and/or the cause of the disease. In the case of cell therapy, the effective amount is an effective dose or number of cells administered to a patient. In some embodiments the patient is a human patient.

As used herein, “disease,” disorder” or “condition” refers to a pathological condition in an organism resulting from cause or condition including, but not limited to, infections, acquired conditions, genetic conditions, and characterized by identifiable symptoms. In particular, it is a condition where treatment is needed and/or desired.

The terms “treating,” “treatment,” or “therapy” of a disease or disorder as used herein mean slowing, stopping or reversing the disease or disorders progression, as evidenced by decreasing, cessation or elimination of either clinical or diagnostic symptoms, by administration of an immunomodulatory protein or engineered cells of the present disclosure either alone or in combination with another compound as described herein. “Treating,” “treatment,” or “therapy” also means a decrease in the severity of symptoms in an acute or chronic disease or disorder or a decrease in the relapse rate as for example in the case of a relapsing or remitting autoimmune disease course or a decrease in inflammation in the case of an inflammatory aspect of an autoimmune disease. “Preventing,” “prophylaxis,” or “prevention” of a disease or disorder as used in the context of this disclosure refers to the administration of an immunomodulatory protein or engineered cells expressing an immunomodulatory protein of the present disclosure, either alone or in combination with another compound, to prevent the occurrence or onset of a disease or disorder or some or all of the symptoms of a disease or disorder or to lessen the likelihood of the onset of a disease or disorder. For example, in the context of cancer, the terms “treatment” or, “inhibit,” “inhibiting” or “inhibition” of cancer refers to at least one of: a statistically significant decrease in the rate of tumor growth, a cessation of tumor growth, or a reduction in the size, mass, metabolic activity, or volume of the tumor, as measured by standard criteria such as, but not limited to, the Response Evaluation Criteria for Solid Tumors (RECIST), or a statistically significant increase in progression free survival (PFS) or overall survival (OS).

As used herein, the terms “delete,” “deletion,” and the like refer to modifications to a gene or a regulatory element associated therewith or operatively linked thereto (e.g., a transcription factor-binding site, such as a promoter or enhancer element) that remove the gene or otherwise render the gene nonfunctional. Exemplary deletions, as described herein, include the removal of the entirety of a nucleic acid encoding a gene of interest, from the start codon to the stop codon of the target gene. Other examples of deletions as described herein include the removal of a portion of the nucleic acid encoding the target gene (e.g., one or more codons, or a portion thereof, such as a single nucleotide deletion) such that, upon expression of the partially-deleted target gene, the product (e.g., RNA transcript, protein product, or regulatory RNA) is nonfunctional or less functional then a wild-type form of the target gene. Exemplary deletions as described herein include the removal of all or a portion of the regulatory element(s) associated with a gene of interest, such as all or a portion of the promoter and/or enhancer nucleic acids that regulate expression of the target gene.

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell).

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell). Exogenous materials include those that are provided from an external source to an organism or to cultured matter extracted there from.

As used herein, the term “percent (%) sequence identity” refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent sequence identity (e.g., gaps can be introduced in one or both of the candidate and reference sequences for optimal alignment and non-homologous sequences can be disregarded for comparison purposes). Alignment for purposes of determining percent sequence identity can be achieved in various ways that are within the skill in the art, for instance, using publicly available computer software, such as BLAST, ALIGN, or Megalign (ONASTAR) software. Those skilled in the art can determine appropriate parameters for measuring alignment, including any algorithms needed to achieve maximal alignment over the full length of the sequences being compared. For example, a reference sequence aligned for comparison with a candidate sequence may show that the candidate sequence exhibits from 50% to 100% sequence identity across the full length of the candidate sequence or a selected portion of contiguous amino acid (or nucleic acid) residues of the candidate sequence. The length of the candidate sequence aligned for comparison purposes may be, for example, at least 30%, (e.g., 30%, 40, 50%, 60%, 70%, 80%, 90%, or 100%) of the length of the reference sequence. When a position in the candidate sequence is occupied by the same amino acid residue as the corresponding position in the reference sequence, then the molecules are identical at that position.

As used herein, the term “operatively linked” in the context of a polynucleotide fragment is intended to mean that the two polynucleotide fragments are joined such that the amino acid sequences encoded by the two polynucleotide fragments remain in-frame.

As used herein, the term “vector” refers to a nucleic acid vector, e.g., a DNA vector, such as a plasmid, a RNA vector, virus or other suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO 1994/11026; incorporated herein by reference. Expression vectors of the disclosure may contain one or more additional sequence elements used for the expression of proteins and/or the integration of these polynucleotide sequences into the genome of a host cell, such as a mammalian cell (e.g., a human cell). Exemplary vectors that can be used for the expression of antibodies and antibody fragments described herein include plasmids that contain regulatory sequences, such as promoter and enhancer regions, which direct gene transcription. Vectors may contain nucleic acids that modulate the rate of translation of a target gene or that improve the stability or nuclear export of the mRNA that results from gene transcription. These sequence elements may include, e.g., 5′ and 3′ untranslated regions, an internal ribosomal entry site (IRES), and polyadenylation signal site in order to direct efficient transcription of the gene carried on the expression vector. The vectors described herein may also contain a polynucleotide encoding a marker for selection of cells that contain such a vector. Examples of a suitable marker include genes that encode resistance to antibiotics, such as ampicillin, chloramphenicol, kanamycin, or nourseothricin. As used herein, the term “VH” refers to the variable region of an immunoglobulin heavy chain of an antibody, including the heavy chain of an Fv, scFv, or Fab. References to “VL” refer to the variable region of an immunoglobulin light chain, including the light chain of an Fv, scFv, dsFv or Fab. Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150,000 Daltons, composed of two identical light (L) chains and two identical heavy (H) chains. Each heavy chain of a native antibody has at the amino terminus a variable domain (VH) followed by a number of constant domains. Each light chain of a native antibody has a variable domain at the amino terminus (VL) and a constant domain at the carboxy terminus.

The term “expression”, as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof.

It is understood that aspects and embodiments of the disclosure described herein include “comprising,” “consisting,” and “consisting essentially of” aspects and embodiments

### II. TRANSGENES

In various embodiments, an oncolytic Orthopoxvirus provided herein is encoded by a nucleic acid containing a recombinant Orthopoxvirus genome and one or more transgenes. In some embodiments, such recombinant Orthopoxviruses are modified in their genomic sequence by the one or more transgenes. In particular embodiments as provided herein, the one or more transgenes are transgenes that are able to inhibit innate and/or adaptive immune responses, such as via activity that results in reduced cytotoxic activity of NK cells and/or T cells against cells infected by the recombinant oncolytic virus. In some embodiments, the one or more transgenes is HCMV glycoprotein UL40 (also termed “UL40) and/or KSHV K5 (also termed “K5”). In some embodiments, provided is an oncolytic Orthopoxvirus encoded by a nucleic acid containing a recombinant Orthopoxvirus genome and a transgene encoding UL40. In some embodiments, provided is an oncolytic Orthopoxvirus encoded by a nucleic acid containing a recombinant Orthopoxvirus genome and a transgene encoding K5. In some embodiments, provided is an oncolytic Orthopoxvirus encoded by a nucleic acid containing a recombinant Orthopoxvirus genome, a transgene encoding K5 and a transgene encoding UL40.

In some embodiments, the exogenous transgenes when expressed with a viral genome, reduces NK cell and T cell activation. In some embodiments, the one or more transgenes encode a protein or proteins to reduce NK cell and/or T cell activation. In some embodiments, the one or more transgenes encode a protein or proteins to reduce NK cell and/or T cell activation and also encode one or more additional transgenes. In some embodiments, the one or more additional transgenes is an immunomodulatory transgene, such as a cytokines, chemokine, tumor microenvironment (TME) modifier, or a bispecific T cell engagers. In some embodiments the additional transgenes are selected from the group consisting of a checkpoint inhibitor, an interleukin, a cytokine. In some embodiments, the one or more transgenes encode a protein or proteins to reduce NK cell and/or T cell activation (e.g. K5 and/or UL40) and an immunomodulatory protein selected from the group consisting of a checkpoint inhibitor, an interleukin, a cytokine. In some embodiments, the one or more transgenes can additionally include an additional NK cell and/or T cell inhibitor.

In some embodiments, the one or more transgenes are exogenous genes that are inserted into the genome. In some embodiments the genome is an Orthopoxvirus genome. In some embodiments, the Orthopoxvirus genome corresponds to any genomes described in section III. In particular embodiments, the Orthopoxvirus genome is a VACV.

In some embodiments, the one or more transgenes is inserted into the 5′ locus of the orthopoxvirus genome in a region. In some embodiments, the locus 5p. Locus 5p is the location of genes C2L, C1L, N1L, N2L, M1L, M2L, K1L, K2L, K3L, K4L, K5L, K6L, K7R, F1L, F2L, and F3L. In some embodiments the one or more transgenes is inserted through homologous recombination. In some embodiments, the homologous recombination is carried out by using a cassette with the one or more transgenes present along with a nucleotide sequence homologous to C2L and a second nucleotide sequence homologous to F3L.

A. UL40 and/or K5 Transgenes

In some embodiments, the one or more transgenes reduces NK cell and T cell activation. In some embodiments the one or more transgenes reduces NK cell activation. In some embodiments, the one or more transgenes reduces T cell activation. In some embodiments, the one or more transgenes reduces NK cell and/or T cell mediated killing of virus infected cells. In some embodiments, the one or more transgenes encode UL40. In some embodiments, the one or more transgenes include UL40 with a sequence put forth in SEQ ID NO:1. In some embodiments, the one or more transgenes encode K5. In some embodiments, the one or more transgenes include K5 with a sequence put forth in SEQ ID NO: 2. In some embodiments, the one or more transgenes encode both UL40 and K5. In some embodiments the one or more transgenes encode UL40 and/or K5 along with other transgenes.

UL40 is a protein encoded by human cytomegalovirus with a representative embodiment of the amino acid sequence set forth in SEQ ID NO: 3 and Uniprot P16780. In some embodiments, UL40 has been inserted into a recombinant Orthopoxvirus genome at the locus 5p. In some embodiments the one or more transgenes is operatively linked to an early or late promoter. In some embodiments, the promoter is H5R. In some embodiments, the promoter is an early H5R promoter. In some embodiments, the promoter is a late H5R promoter. In some embodiments the promoter has a sequence set forth in SEQ ID NO: 4. In some embodiments, the promoter is p7.5. In some embodiments, the promoter has a sequence set forth in SEQ ID NO: 5. UL40 contains an HLA-E binding peptide in its leader sequence (Prod'homme et al. (2012) J Immunol. 188:2794-2804, and Ulbrecht et al. (2000) J Immunol 164:5019-5022) which upon binding to HLA-E promotes cell surface expression of HLA-E (Tomasec et al. (2000) Science 287:1031-1033). Binding of HLA-E/UL40 peptide complex to the inhibitory NK surface receptor NKG2A/CD94 promotes efficient protection against lysis mediated by NKG2A/CD94 NK cells (Ulbrecht et al. (2000) J Immunol 164:5019-5022, Tomasec et al. (2000) Science 287:1031-1033, Wang et al. (2002) Proc National Acad Sci. 99:7570-7575, Borrego et al. (1998) J Exp Medicin. 187:813-818, Braud et al. (1998) Nature. 391:795-799, and Llano et al. (1998) Proc National Acad Sci. 95:5199-5204). Unexpectedly, this disclosure contains what is believed to be the first report of UL40 protecting virus-infected cells against killing mediated by T cells. NKG2A/CD94 can be expressed on a subset of CD8⁺ T cells.

In some embodiments UL40 refers to a cytomegalovirus (e.g., human cytomegalovirus) gene, such as a gene that encodes a protein that contains an HLA-E ligand which promotes cell surface expression of HLA-E. A nonlimiting example of a protein sequence encoded by an exemplary UL40 gene in is given in UniProtKB database entry P16780 and is reproduced below:

(SEQ ID NO: 3)
MNKFSNTRIGFTCAVMAPRTLILTVGLLCMRIRSLLCSPAETTVTTAAV
TSAHGPLCPLVFQGWAYAVYHQGDMALMTLDVYCCRQTSNNTVVAFSHH
PADNTLLIEVGNNTRRHVDGISCQDHFRAQHQDCPAQTVHVRGVNESAF
GLTHLQSCCLNEHSQLSERVAYHLKLRPATFGLETWAMYTVGILALGSF
SSFYSQIARSLGVLPNDHHYALKKA.

K5 is a protein encoded by Kaposi's sarcoma-associated herpesvirus with a representative embodiment of the amino acid sequence set forth in SEQ ID NO 6. In some embodiments, K5 has been inserted into a recombinant Orthopoxvirus genome at the locus 5p. In some embodiments the one or more transgenes is operatively linked to an early or late promoter. In some embodiments, the promoter is H5R. In some embodiments, the promoter is an early H5R promoter. In some embodiments, the promoter is a late H5R promoter. In some embodiments the promoter has a sequence set forth in SEQ ID NO: 4. In some embodiments, the promoter is p7.5. In some embodiments, the promoter has a sequence set forth in SEQ ID NO: 5. K5 is a multifunctional protein that inhibits killing mediated by both NK and T cells by promoting endocytosis of MHC class I molecules, NKG2D ligands, and ICAM_1 on infected cells which protect infected cells from killing mediated by both NK and T cells.

In some embodiments, K5 refers to a Rhadinovirus (e.g., Kaposi's sarcoma-associated herpesvirus) gene, such as a gene that encodes a ubiquitin ligase protein which downregulates cell surface expression of MHC-I molecules and NKG2D ligands. A nonlimiting example of a protein sequence encoded by an exemplary K5 gene in is given in UniProtKB database entry F5H9K4 and is reproduced below:

(SEQ ID NO: 6)
MASKDVEEGVEGPICWICREEVGNEGIHPCACTGELDVVHPQCLSTWLT
VSRNTACQMCRVIYRTRTQWRSRLNLWPEMERQEIFELFLLMSVVVAGL
VGVALCTWTLLVILTAPAGTFSPGAVLGFLCFFGFYQIFIVFAFGGICR
VSGTVRALYAANNTRVTVLPYRRPRRPTANEDNIELTVLVGPAGGTDEE
PTDESSEGDVASGDKERDGSSGDEPDGGPNDRAGLRGTARTDLCAPTKK
PVRKNHPKNNG.

### B. Additional Transgenes

In some embodiments, additional transgenes can be encoded in the nucleic acid. These transgenes can encode proteins that are or are used to deliver virus trackers, checkpoint inhibitors, cytokines, interleukins, immunomodulatory agents, and therapeutic agents. In some embodiments, immunomodulatory agents are selected from: FMS-like tyrosine kinase 3 ligand (FLT3L), an antibody that specifically binds to CTLA-4, and an Interleukin 12 (IL-12) polypeptide, optionally wherein the IL-12 polypeptide is a membrane-bound IL-12. In some embodiments, the additional transgenes encode one or more of the following: FLT3L, CTLA-4, and an IL-12 polypeptide, optionally wherein the IL-12 polypeptide is a membrane-bound IL-12. In some embodiments, the additional transgenes encode all of the following: FLT3L, CTLA-4, and an IL-12 polypeptide, optionally wherein the IL-12 polypeptide is a membrane-bound IL-12.

In some embodiments, the additional transgenes contain a nucleic acid that encodes a checkpoint inhibitor. In some embodiments, the checkpoint inhibitor is an antibody that binds to CTLA-4. In some embodiments, the additional transgenes contain a nucleic acid which encodes an anti-CTLA-4 antibody or antigen-binding fragment thereof. In some embodiments the additional transgene is a nucleic acid encoding a gene for an anti-CTLA-4 antibody or antigen-binding fragment thereof and a promoter operably linked to the gene. In specific embodiments, the anti-CTLA-4 antibody or antigen-binding fragment thereof encoded by the nucleic acid sequence comprises the 6 complementarity-determining regions (CDRs) of ipilimumab. In specific embodiments, the gene encoded by the nucleic acid sequence encodes an amino acid sequence comprising the amino acid sequence set forth in SEQ ID NO: 7. In specific embodiments, the nucleic acid sequence comprises the sequence set forth in SEQ ID NO: 8.

In some embodiments, the additional transgenes contain a nucleic acid that encodes an interleukin. In some embodiments, the interleukin is IL-12. In some embodiments, the IL-12 is membrane bound. In some embodiments, the IL-12 is IL-12p35. In specific embodiments, the IL-12 polypeptide is membrane-bound. In specific embodiments, the IL-12 polypeptide comprises IL-12 p35 (e.g., human IL-12 p35), IL-12 p40 (e.g., human IL-12 p40) or IL-12 p70 (e.g., human IL-12 p70). In specific embodiments, the IL-12 polypeptide is membrane-bound and comprises IL-12 p35 (e.g., human IL-12 p35), or IL-12 p70 (e.g., human IL-12 p70), and a transmembrane domain and a cytoplasmic domain (e.g., the transmembrane and cytoplasmic domains of B7-1, TNFa, or FLT3L). In specific embodiments, the IL-12 polypeptide comprises the amino acid sequence set forth in SEQ ID NO: 9. In specific embodiments, the second nucleotide sequence comprises the sequence set forth in SEQ ID NO: 10. In specific embodiments, the IL-12 polypeptide is IL-12 p40 and comprises the amino acid sequence set forth in SEQ ID NO: 11. In specific embodiments, the second nucleotide sequence comprises the sequence set forth in SEQ ID NO: 12.

In some embodiments the additional transgenes contain a nucleic acid that encodes a cytokine. In some embodiments the cytokine is an Flt3 ligand (FLT3L). In some embodiments the Flt3 ligand is soluble. In particular embodiments, the FLT3L is a soluble form of human FLT3L. In certain embodiments, the FLT3L is a soluble form of the human FL T3L set forth in GenBank Accession No. U03858. 1. For example, in specific embodiments, the FLT3L lacks the entire FLT3L transmembrane (e.g., the transmembrane domain of the human FLT3L set forth in GenBank Accession No. U03858.1) In other examples, the FLT3L sequence lacks at least 80%, at least 85%, at least 90%, or at least 95% of the FLT3L transmembrane domain (e.g., the transmembrane domain of the human FLT3L set forth in GenBank Accession No. U03858.1). In one embodiment, the FLT3L sequence lacks the entire FLT3L transmembrane domain and the entire FLT3L cytoplasmic domain. In another embodiment, the FLT3L sequence lacks at least 80%, at least 85%, at least 90%, or at least 95% of the FLT3L transmembrane domain and the entire FLT3L cytoplasmic domain. In another embodiment, the FLT3L sequence lacks at least 80%, at least 85%, at least 90%, or at least 95% of the FLT3L transmembrane domain and at least 25%, at least 30%, at least 40%, at least 50%, at least 60%, at least 70%, at least 80%, at least 85%, at least 90%, or at least 95% of the FLT3L cytoplasmic domain. In another embodiment, the FLT3L sequence lacks the entire FLT3L transmembrane domain and 5, 6, 7, 8, 9, 10, 12, 13, 14, or 15 of the N-terminal amino acid residues of the FLT3L cytoplasmic domain. In another embodiment, the FLT3L sequence lacks the entire FLT3L transmembrane domain and 1, 2, 3, or 4 of the N-terminal amino acid residues of the FLT3L cytoplasmic domain. In certain of the embodiments and aspects, the transmembrane and cytoplasmic domains are of the FLT3L sequence set forth in GenBank Accession No. U03858.1.

In some embodiments, the additional transgenes can include virus trackers. In some embodiments, the virus trackers are a fluorescent protein. In some embodiments the fluorescent protein is an RFP. In some embodiment, the fluorescent protein is GFP. In some embodiments, the GFP is encoded by a sequence as set forth in SEQ ID NO: 13. In some embodiment, the additional transgene is operatively linked to a promoter. In some embodiments, the promoter is an early/late promoter. In some embodiments, the promoter is a E3L promoter. In some embodiments, the promoter sequence is set forth in SEQ ID NO: 14.

### III. RECOMBINANT ORTHOPOXVIRUS

In some embodiments, a nucleic acid encodes a recombinant Orthopoxvirus genome such that the nucleic acid is capable of producing an Orthopoxvirus which can in turn infect cells. In some embodiments, the Orthopoxvirus targets tumor cells. In some embodiments, an Orthopoxvirus is generated using a nucleic acid described in this disclosure. In some embodiments, an Orthopoxvirus is generated from a nucleic acid which contains a recombinant Orthopoxvirus genome and one or more transgenes. In some embodiments, an Orthopoxvirus is generated from a nucleic acid which contains a recombinant Orthopoxvirus genome and one or more transgenes containing (a) nucleotide sequence encoding UL40 and/or (b) a nucleotide sequence encoding K5. In some embodiments, an Orthopoxvirus generated from a nucleic acid disclosed herein, can be used to treat cancer in a subject. In some embodiments, the Orthopoxvirus is a Vaccinia virus. In some embodiments, the Vaccinia virus is a recombinant Vaccinia virus generated from a Copenhagen strain of Vaccinia virus.

In some embodiments, the nucleic acid contains an Orthopoxvirus genome with a nucleotide sequence at least 80%, at least 85%, at least 90%, or at least 95% similar to the sequence set forth in SEQ ID NO:15 In some embodiments, the Orthopoxvirus is a parental SKV and is encoded in the sequence set forth in SEQ ID NO: 15. In some embodiments, an Orthopoxvirus is encoded in the sequence set forth in SEQ ID NO: 15 with one or more transgenes described in section II added.

In some embodiments, the Orthopoxvirus is SKV-UL40. In some embodiments, SKV-UL40 is encoded by the sequence set forth in SEQ ID NO: 16.

In some embodiments, the Orthopoxvirus is SKV-K5. In some embodiments, SKV-K5 is encoded by the sequence set forth in SEQ ID NO: 17.

In some embodiments, the Orthopoxvirus is SKV-UL40_K5. In some embodiments, SKV-UL40_K5 is encoded by the sequence set forth in SEQ ID NO: 18.

### A. Orthopox and Vaccinia Viruses

Vaccinia virus is a member of the poxvirus or Poxviridae family, the Chordopoxyirinae subfamily, and the Orthopoxvirus genus. Orthopoxvirus is relatively more homogeneous than other members of the Chordopoxyirinae subfamily and includes 11 distinct but closely related species, which includes vaccinia virus, variola virus (causative agent of smallpox), cowpox virus, buffalopox virus, monkeypox virus, mousepox virus and horsepox virus species as well as others (see Moss, 1996).

Vaccinia virus is a large, complex enveloped virus having a linear double-stranded DNA genome of about 190 kb and encoding approximately 250 genes. Vaccinia is well-known for its role as a vaccine that eradicated smallpox. Post-eradication of smallpox, scientists have been exploring the use of vaccinia as a tool for delivering genes into biological tissues (gene therapy and genetic engineering). Vaccinia virus is unique among DNA viruses as it replicates only in the cytoplasm of the host cell. Therefore, a large genome is required to encode various enzymes and proteins needed for viral DNA replication. During replication, vaccinia produces several infectious forms, which differ in their outer membranes: the intracellular mature virion (IMV), the intracellular enveloped virion (IEV), the cell-associated enveloped virion (CEV), and the extracellular enveloped virion (EEV). IMV is the most abundant infectious form and is thought to be responsible for spread between hosts. On the other hand, the CEV is believed to play a role in cell-to-cell spread, and the EEV is thought to be important for long range dissemination within the host organism.

Vaccinia virus is closely related to the virus that causes cowpox. The precise origin of vaccinia is unknown, but the most common view is that vaccinia virus, cowpox virus, and variola virus (the causative agent for smallpox) were all derived from a common ancestral virus. There is also speculation that vaccinia virus was originally isolated from horses. A vaccinia virus infection is mild and typically asymptomatic in healthy individuals, but it may cause a mild rash and fever, with an extremely low rate of fatality. An immune response generated against a vaccinia virus infection protects that person against a lethal smallpox infection. For this reason, vaccinia virus was used as a live-virus vaccine against smallpox. The vaccinia virus vaccine is safe because it does not contain the smallpox virus, but occasionally certain complications and/or vaccine adverse effects may arise, especially if the vaccine is immunocompromised.

Exemplary strains of the vaccinia virus include, but are not limited to, Copenhagen, Western Reserve, Wyeth, Lister, EM63, ACAM2000, LC16m8, CV-1, modified vaccinia Ankara (MVA), Dairen I, GLV-1h68, IHD-J, L-IVP, LC16m8, LC16mO, Tashkent, Tian Tan, and W AU86/88-1.

In one aspect, provided herein is a nucleic acid comprising an Orthopoxvirus genome and one or more transgenes. In some embodiments, the one or more transgenes are inserted into the Orthopox virus genome, creating one nucleic acid. In some embodiments, the Orthopoxvirus genome is a Vaccinia virus genome. In some embodiments, the Vaccinia virus genome is from a strain selected from Copenhagen, Western Reserve, Wyeth, Lister, EM63, ACAM2000, LC16m8, CV-1, modified vaccinia Ankara (MVA), Dairen I, GLV-1h68, IHD-J, L-IVP, LC16m8, LC16mO, Tashkent, Tian Tan, and W AU86/88-1, or is an attenuated version of any of the foregoing. In some embodiments, the Vaccinia virus genome is from a Copenhagen, TianTan, Lister, Wyeth, or Western Reserve strain or is a recombinant attenuated strain thereof. In some embodiments the Orthopoxvirus genome is used as a backbone for inserting one or more transgenes into.

In some embodiments, the Orthopoxvirus genome is a recombinant Orthopoxvirus genome is an attenuated strain that is attenuated by modification to delete various genes to enhance the oncolytic activity of the orthopoxvirus. In some embodiments, the deletions are deletions of genes that are either involved in blocking a host response to viral infection or otherwise have an unknown function.

In some embodiments, the Vaccinia virus is derived from a Copenhagen (Cop) strain, such as a Copenhagen strain that is a modified attenuated strain. In some embodiments, the attenuated Copenhagen strain is deleted genes that include deletion of one or more of C1L, N1L, N2L, M1L, M2L, K1L, K2L, K3L, K4L, K5L, K6L, K7R, F1L, F2L, F3L, B14R, B15R, B16R, B17L, B18R, B20R, B21R, B22R, B23R, B24R, B25R, B26R, B27R, B28R, B29R and B8R. In some embodiments, at least 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 of the above genes are deleted from the recombinant orthopox genome. In some embodiments, all of the above genes are deleted from the recombinant orthopoxvirus genome.

In some embodiments, B8R refers to an Orthopoxvirus (e.g., vaccinia, e.g., Copenhagen) gene, such as a gene that encodes a secreted protein with homology to the gamma interferon (IFN-7) receptor. A nonlimiting example of a protein sequence encoded by an exemplary B8R gene in a Copenhagen strain of the vaccinia virus is given in UniProtKB database entry P21004 and a nonlimiting example of a nucleic acid sequence is set forth in SEQ ID NO:19.

The B8R may also include fragments or variants of the protein listed above, or of homologous genes from another vaccinia virus strain. Variants include, without limitation, those sequences having 85 percent or greater identity to the sequences disclosed herein.

In some embodiments, B14R refers to an Orthopoxvirus (e.g., vaccinia, e.g., Copenhagen) gene. An example of a protein sequence encoded by an exemplary B14R gene in a Copenhagen strain of the vaccinia virus is given in UniProtKB database entry P20842 and a nonlimiting example of a nucleic acid sequence is set forth in SEQ ID NO: 20.

In some embodiments, B15R refers to an Orthopoxvirus (e.g., vaccinia, e.g., Copenhagen) gene. An example of a protein sequence encoded by an exemplary B15R gene in a Copenhagen strain of the vaccinia virus is given in UniProtKB database entry P21089 and a nonlimiting example of a nucleic acid sequence is set forth in SEQ ID NO: 21.

In some embodiments, B16R refers to an Orthopoxvirus (e.g., vaccinia, e.g., Copenhagen) gene, such as a gene that encodes a IL-1-beta inhibitor. An example of a protein sequence encoded by an exemplary B16R gene in a Copenhagen strain of the vaccinia virus is given in UniProtKB database entry P21116 and a nonlimiting example of a nucleic acid sequence is set forth in SEQ ID NO: 22.

In some embodiments, B17L refers to an Orthopoxvirus (e.g., vaccinia, e.g., Copenhagen) gene. An example of a protein sequence encoded by an exemplary B17L gene in a Copenhagen strain of the vaccinia virus is given in UniProtKB database entry P21075 and a nonlimiting example of a nucleic acid sequence is set forth in SEQ ID NO: 23.

In some embodiments, B18R refers to an Orthopoxvirus (e.g., vaccinia, e.g., Copenhagen) gene, such as a gene that encodes an Ankyrin repeat protein. An example of a protein sequence encoded by an exemplary B18R gene in a Copenhagen strain of the vaccinia virus is given in UniProtKB database entry P21076 and a nonlimiting example of a nucleic acid sequence is set forth in SEQ ID NO: 24.

In some embodiments, B19R refers to an Orthopoxvirus (e.g., vaccinia, e.g., Copenhagen) gene, such as a gene that encodes a IFN-alpha-beta-receptor-like secreted glycoprotein. An example of a protein sequence encoded by an exemplary B19R gene in a Copenhagen strain of the vaccinia virus is given in UniProtKB database entry P21077 and a nonlimiting example of a nucleic acid sequence is set forth in SEQ ID NO: 25.

In some embodiments, B20R refers to an Orthopoxvirus (e.g., vaccinia, e.g., Copenhagen) gene, such as a gene that encodes an Ankyrin repeat protein. An example of a protein sequence encoded by an exemplary B20R gene in a Copenhagen strain of the vaccinia virus is given in UniProtKB database entry P21078 and a nonlimiting example of a nucleic acid sequence is set forth in SEQ ID NO: 26.

In some embodiments, C1L refers to an Orthopoxvirus (e.g., vaccinia, e.g., Copenhagen) gene. An example of a protein sequence encoded by an exemplary C1L gene in a Copenhagen strain of the vaccinia virus is given in UniProtKB database entry P21036 and a nonlimiting example of a nucleic acid sequence is set forth in SEQ ID NO: 27.

In some embodiments, C2L refers to an Orthopoxvirus (e.g., vaccinia, e.g., Copenhagen) gene, such as a gene that encodes a kelch-like protein that affects calcium-independent adhesion to the extracellular matrix. An example of a protein sequence encoded by an exemplary C2L gene in a Copenhagen strain of the vaccinia virus is given in UniProtKB database entry P21037 and a nonlimiting example of a nucleic acid sequence is set forth in SEQ ID NO: 28.

In some embodiments, F1L refers to a Orthopoxvirus (e.g., vaccinia, e.g., Copenhagen) gene, such as a gene that encodes a caspase-9 inhibitor. An example of a protein sequence encoded by an exemplary F1L gene in a Copenhagen strain of the vaccinia virus is given in UniProtKB database entry P68450 and a nonlimiting example of a nucleic acid sequence is set forth in SEQ ID NO: 29.

In some embodiments, F2L refers to an Orthopoxvirus (e.g., vaccinia, e.g., Copenhagen) gene, such as a gene that encodes a deoxyuridine triphosphatase (dUTPase). An example of a protein sequence encoded by an exemplary F2L gene in a Copenhagen strain of the vaccinia virus is given in UniProtKB database entry P68634 and a nonlimiting example of a nucleic acid sequence is set forth in SEQ ID NO: 30.

In some embodiments, F3L refers to an Orthopoxvirus (e.g., vaccinia, e.g., Copenhagen) gene, such as a gene that encodes a kelch-like protein that is an innate immune response modifier and a virulence factor. An example of a protein sequence encoded by an exemplary F3L gene in a Copenhagen strain of the vaccinia virus is given in UniProtKB database entry P21013 and a nonlimiting example of a nucleic acid sequence is set forth in SEQ ID NO: 31.

In some embodiments, K1L refers to an Orthopoxvirus (e.g., vaccinia, e.g., Copenhagen) gene, such as a gene that encodes an NF-KB inhibitor. An example of a protein sequence encoded by an exemplary K1L gene in a Copenhagen strain of the vaccinia virus is given in UniProtKB database entry P20632 and a nonlimiting example of a nucleic acid sequence is set forth in SEQ ID NO: 32.

In some embodiments, K2L refers to an Orthopoxvirus (e.g., vaccinia, e.g., Copenhagen) gene, such as a gene that encodes a serine protease inhibitor that prevents cell fusion. An example of a protein sequence encoded by an exemplary K2L gene in a Copenhagen strain of the vaccinia virus is given in UniProtKB database entry P20532 and a nonlimiting example of a nucleic acid sequence is set forth in SEQ ID NO: 33.

In some embodiments, K3L refers to an Orthopoxvirus (e.g., vaccinia, e.g., Copenhagen) gene, such as a gene that encodes a PKR inhibitor. An example of a protein sequence encoded by an exemplary K3L gene in a Copenhagen strain of the vaccinia virus is given in UniProtKB database entry P20639 and a nonlimiting example of a nucleic acid sequence is set forth in SEQ ID NO: 34.

In some embodiments, K4L refers to an Orthopoxvirus (e.g., vaccinia, e.g., Copenhagen) gene, such as a gene that encodes a DNA modifying nuclease (e.g., DNA nicking enzyme). An example of a protein sequence encoded by an exemplary K4L gene in a Copenhagen strain of the vaccinia virus is given in UniProtKB database entry P20537 and a nonlimiting example of a nucleic acid sequence is set forth in SEQ ID NO: 35.

In some embodiments, K5L refers to an Orthopoxvirus (e.g., vaccinia, e.g., Copenhagen) gene, such as a gene that encodes a putative monoglyceride lipase. An example of a protein sequence encoded by an exemplary K5L gene in a Copenhagen strain of the vaccinia virus is given in UniProtKB database entry P21084 and a nonlimiting example of a nucleic acid sequence is set forth in SEQ ID NO: 36.

In some embodiments, K6L refers to an Orthopoxvirus (e.g., vaccinia, e.g., Copenhagen) gene, such as a gene that encodes a putative monoglyceride lipase. An example of a protein sequence encoded by an exemplary K6L gene in a Copenhagen strain of the vaccinia virus is given in UniProtKB database entry P68465 and a nonlimiting example of a nucleic acid sequence is set forth in SEQ ID NO: 37.

In some embodiments, K7R refers to an Orthopoxvirus (e.g., vaccinia, e.g., Copenhagen) gene, such as a gene that encodes an inhibitor of NF-κB and IRF3. An example of a protein sequence encoded by an exemplary K7R gene in a Copenhagen strain of the vaccinia virus is given in UniProtKB database entry P68467 and a nonlimiting example of a nucleic acid sequence is set forth in SEQ ID NO: 38.

In some embodiments, M1L refers to an Orthopoxvirus (e.g., vaccinia, e.g., Copenhagen) gene, such as a gene that encodes an Ankyrin repeat protein. An example of a protein sequence encoded by an exemplary M1L gene in a Copenhagen strain of the vaccinia virus is given in UniProtKB database entry P20640 and a nonlimiting example of a nucleic acid sequence is set forth in SEQ ID NO: 39.

In some embodiments, M2L refers to an Orthopoxvirus (e.g., vaccinia, e.g., Copenhagen) gene, such as a gene that encodes an inhibitor of NF-KB and apoptosis. An example of a protein sequence encoded by an exemplary M2L gene in a Copenhagen strain of the vaccinia virus is given in UniProtKB database entry Q1PJ18 and a nonlimiting example of a nucleic acid sequence is set forth in SEQ ID NO: 40.

In some embodiments, N1L refers to an Orthopoxvirus (e.g., vaccinia, e.g., Copenhagen) gene, such as a gene that encodes a BCL-2-like protein that inhibits NF-κB and apoptosis. An example of a protein sequence encoded by an exemplary N1L gene in a Copenhagen strain of the vaccinia virus is given in UniProtKB database entry P21054 and a nonlimiting example of a nucleic acid sequence is set forth in SEQ ID NO: 41.

In some embodiments, N2L refers to an Orthopoxvirus (e.g., vaccinia, e.g., Copenhagen) gene, such as a gene that encodes an inhibitor of IRF3. An example of a protein sequence encoded by an exemplary N2L gene in a Copenhagen strain of the vaccinia virus is given in UniProtKB database entry P20641 and a nonlimiting example of a nucleic acid sequence is set forth in SEQ ID NO: 42.

In some embodiments, the Orthopoxvirus genome to which the one or more transgenes (e.g. K5 and/or UL40) is inserted includes any as described in WO2019134048, WO2019134049, WO2020124273, and WO2020124274, each herein incorporated by reference in their entirety. In some embodiments, the Orthopox genome is designated CopMD5p, CopMD3p or CopMD5p3p. In some embodiments, CopMD5p represents a clone, which was found to harbor major genomic deletions in 5′ genes (deletions in representative 5′ genes C2L, C1L, N1L, N2L, MIL, M2L, K1L, K2L, K3L, K4L, K5L, K6L, K7R, F1L, F2L, F3L). In some embodiments, CopMD3p represents a clone with deletions in 3′ genes (e.g. B14R, B15R, B16R, B17L, B18R, B19R, and B20R) as well as deletions in each of the B21R, B22R, B23R, B24R, B25R, B26R, B27R, B28R, and B29R copies of ITRs. CopMD5p3p is a double deleted genome which contains both 5′ gene deletions and 3′ gene deletions as well as the deletions in ITR genes such that the genome has deletions in the C2L, C1L, N1L, N2L, MIL, M2L, K1L, K2L, K3L, K4L, K5L, K6L, K7R, FIL, F2L, F3L, B14R, B15R, B16R, B17L, B18R, B19R, and B20R genes, as well as deletions in each of the B21R, B22R, B23R, B24R, B25R, B26R, B27R, B28R, and B29R copies of ITRs

In some embodiments, the Orthopoxvirus genome is a Vaccinia virus genome that further contains deletions of the B8R gene. B8R gene encodes a secreted protein with homology to gamma interferon receptor (IFN-g). In vitro, the B8R protein binds to and neutralizes the antiviral activity of several species of gamma interferon including human and rat gamma interferon; it does not, however, bind significantly to murine IFN-g. In some embodiments, the recombinant Vaccinia virus to which the one or more transgenes (e.g. K5 and/or UL40) are inserted lacks the B8R gene or otherwise has a knock of a portion of B8R gene effectively preventing expression of the gene.

In some embodiments, the Vaccinia virus genome is a Superior Killing Virus (SKV), an attenuated vector derived from the Copenhagen strain of Vaccinia virus that contains two (2) major deletions at the 5′ and 3′ ends as well as the deletion of the B8R gene as described in (see e.g., WO2020124274 which is incorporated by reference in its entirety). Some embodiments provide a nucleic acid comprising an Orthopoxvirus genome comprising: (a) deletions in the one or more of the following genes: C2L, C1L, N1L, N2L, M1L, M2L, K1L, K2L, K3L, K4L, K5L, K6L, K7R, F1L, F2L, F3L, B14R, B15R, B16R, B17L, B18R, and B20R; (b) deletions in one or more of the following genes in the 3′ inverted terminal repeat (ITR): B21R, B22R, B23R, B24R, B25R, B26R, B27R, B28R, and B29R; and/or (c) deletion in the B8R gene. In some embodiments, the one or more transgenes (e.g. K5 and/or UL40) is inserted into such a Vaccinia virus genome.

In some embodiments, a gene deletion removes the entire sequence of the gene. In other embodiments, a gene deletion is a partial deletion, that is, one that removes part of the sequence of the gene. In one embodiment, a gene deletion is a partial deletion that removes at least 30%, at least 40%, at least 50%, at least 60%, at least 70%, at least 80%, at least 90% or at least 95% of the sequence of the gene. In one embodiment, a gene deletion is a partial deletion that removes at least 30%, at least 40%, at least 50%, at least 60%, at least 70%, at least 80%, or at least 90% of the protein coding sequence of the gene. In other embodiments, a gene deletion removes 100% of the sequence of the gene. In yet other embodiments, a gene deletion removes 100% of the protein coding sequence of the gene. In one embodiment, a gene deletion removes at least 50, at least 100, at least 200, at least 300, at least 400, at least 500, at least 600, at least 700, at least 800, at least 900, or at least 1000 nucleotides of the sequence of the gene. In another embodiment, a gene deletion is a partial deletion that removes at least 50, at least 100, at least 200, at least 300, at least 400, at least 500, at least 600, at least 700, at least 800, at least 900, or at least 1000 nucleotides of the sequence of the gene. In a specific embodiment, a partial deletion in a gene results in a partial gene.

In some embodiments, the Orthopoxvirus genome is a Vaccinia virus genome nucleotide sequence that is at least 80%, at least 85%, at least 90%, or at least 95% similar to SEQ ID NO:15. In some embodiments, the Orthopoxvirus genome is a Vaccinia virus nucleotide sequence set forth in SEQ IN NO: 13. In some embodiments, the one or more transgenes (e.g. K5 and/or UL40) is inserted into a Vaccinia virus genome nucleotide sequence that is at least 80%, at least 85%, at least 90%, or at least 95% similar to SEQ ID NO:15. In some embodiments, the one or more transgenes (e.g. K5 and/or UL40) is inserted into a Vaccinia virus genome nucleotide sequence set forth in SEQ IN NO: 13.

In some embodiments, one or more transgenes are inserted into a region of the genome at the 5′ end, such as replacing or inserted in a locus of a deleted gene or inserted between two partially deleted gene. In some embodiments, transgenes are inserted between the partial C2L and F3L vaccinia genes (that is, is present between the partial C2L and F3L genes). In some embodiments, a transgene is inserted into the locus of the deletion in the B8R gene (that is, are present in the locus of the deletion in the B8R gene).

In some embodiments, the UL40 transgene is inserted between the portion of the C2L vaccinia gene that remains and the portion of the F3L vaccinia gene that remains (that is, is present between the portion of the C2L vaccinia gene that remains and the portion of the F3L vaccinia gene that remains). In other embodiments, the UL40 transgene is inserted into the locus of the deletion in the B8R gene (that is, are present in the locus of the deletion in the B8R gene).

In some embodiments, the K5 transgene is inserted between the portion of the C2L vaccinia gene that remains and the portion of the F3L vaccinia gene that remains (that is, is present between the portion of the C2L vaccinia gene that remains and the portion of the F3L vaccinia gene that remains). In other embodiments, the K5 transgene is inserted into the locus of the deletion in the B8R gene (that is, are present in the locus of the deletion in the B8R gene).

In some embodiments, the UL40 transgene is inserted between the portion of the C2L vaccinia gene that remains and the portion of the F3L vaccinia gene that remains (that is, is present between the portion of the C2L vaccinia gene that remains and the portion of the F3L vaccinia gene that remains), and the K5 transgene is inserted into the locus of the deletion in the B8R gene (that is, are present in the locus of the deletion in the B8R gene).

In some embodiments, the K5 transgene is inserted between the portion of the C2L vaccinia gene that remains and the portion of the F3L vaccinia gene that remains (that is, is present between the portion of the C2L vaccinia gene that remains and the portion of the F3L vaccinia gene that remains), and the UL40 transgene is inserted into the locus of the deletion in the B8R gene (that is, are present in the locus of the deletion in the B8R gene).

In some embodiments, one or more third transgene (e.g. any of the additional transgenes described above) is also inserted into a region of the genome at the 5′ end of the orthopoxvirus (e.g. Vaccinia virus) genome, such as replacing or insertion in a locus of a deleted gene or inserted between two partially deleted gene. In some embodiments, a third transgene is inserted into the locus of the deletion in the B8R gene (that is, are present in the locus of the deletion in the B8R gene). In some cases, at least two transgenes (e.g. UL40 or K5, and the one or more additional transgene) is inserted into the locus of the deletion in the B8R gene (that is, are present in the locus of the deletion in the B8R gene).

In some embodiments, the nucleic acid containing a recombinant Orthopoxvirus genome and the one or more transgenes (e.g. UL40) includes a sequence that is at least 80%, at least 85%, at least 90%, or at least 95% similar to the sequence set forth in SEQ ID NO: 16. In some embodiments, the nucleic acid containing a recombinant Orthopoxvirus genome and the one or more transgenes (e.g. UL40) includes the sequence set forth in SEQ ID NO: 16.

In some embodiments, the nucleic acid containing a recombinant Orthopoxvirus genome and the one or more transgenes (e.g. K5) includes a sequence that is at least 80%, at least 85%, at least 90%, or at least 95% similar to the sequence set forth in SEQ ID NO: 17. In some embodiments, the nucleic acid containing a recombinant Orthopoxvirus genome and one or more transgenes (e.g. K5) includes the sequence set forth in SEQ ID NO: 17.

In some embodiments, the nucleic acid containing a recombinant Orthopoxvirus genome and the one or more transgenes (e.g. K5 and UL40) includes a sequence that is at least 80%, at least 85%, at least 90%, or at least 95% similar to the sequence set forth in SEQ ID NO: 18. In some embodiments, the nucleic acid containing a recombinant Orthopoxvirus genome and one or more transgenes contains the sequence set forth in SEQ ID NO: 18 (e.g. K5 and UL40).

### B. Cells, Cell Lines, and Packaging Cell Lines and Methods of Virus Propagation

In some embodiments, the provided recombinant orthopoxviruses, including those constructed with one or more gene deletions compared to wild-type or starting virus strain, can be generated by various well known methods. This section summarizes various protocols, by way of example, for producing recombinant orthopoxviruses described herein, such as methods for generating mutated viruses through the use of recombinant DNA technology.

For example, to generate mutations in the orthopoxvirus genome, native and modified polypeptides may be encoded by a nucleic acid molecule comprised in a vector. Vectors may include, for example, plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al, (1989) and Ausubel et al, 1994, both incorporated herein by reference in their entirety. In addition to encoding a modified polypeptide, a vector may encode non-modified polypeptide sequences such as a tag or targeting molecule.

In order to propagate a vector in a host cell, it may contain one or more origins of replication sites (often termed“ori”), which is a specific nucleic acid sequence at which replication is initiated. Alternatively, an autonomously replicating sequence (ARS) can be employed if the host cell is yeast.

In the context of expressing a heterologous nucleic acid sequence, “host cell” refers to a prokaryotic or eukaryotic cell, and it includes any transformable organism that is capable of replicating a vector and/or expressing a heterologous gene encoded by a vector. A host cell can, and has been, used as a recipient for vectors or viruses (which qualify as a vector if they express an exogenous polypeptide). A host cell may be “transfected” or “transformed,” which refers to a process by which exogenous nucleic acid, such as a modified protein-encoding sequence, is transferred or introduced into the host cell. A transformed cell includes the primary subject cell and its progeny. Host cells may be derived from prokaryotes or eukaryotes, including yeast cells, insect cells, and mammalian cells, depending upon whether the desired result is replication of the vector or expression of part or all of the vector-encoded nucleic acid sequences. Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors.

Bacterial cells used as host cells for vector replication and/or expression include DH5a, JM109, and KCB, as well as a number of commercially available bacterial hosts such as SURE® Competent Cells and SOLOPACK™ Gold Cells (STRATAGENE®, La Jolla, Calif.). Alternatively, bacterial cells such as E. coli LE392 could be used as host cells for phage viruses. Appropriate yeast cells include Saccharomyces cerevisiae, Saccharomyces pombe, and Pichia pastoris. Examples of eukaryotic host cells for replication and/or expression of a vector include HeLa, NIH3T3, Jurkat, 293, COS, CHO, Saos, and PC 12.

Many host cells from various cell types and organisms are available and would be known to one of skill in the art. Similarly, a viral vector may be used in conjunction with either a eukaryotic or prokaryotic host cell, particularly one that is permissive for replication or expression of the vector. Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

In one aspect, provided herein is a cell containing the nucleic acid described in any previous section. In another aspect, provided herein is a cell comprising the virus described in any previous section. In certain embodiments, the cell provided herein is a mammalian cell (e.g., a human cell). In certain embodiments, the cell provided herein is a host cell.

In one aspect, provided herein is a cell line comprising the nucleic acid described in any previous section. In another aspect, provided herein is a cell line comprising the virus described in any previous section. In certain embodiments, the cell line provided herein is a mammalian cell line (e.g., a human cell line).

In one aspect, provided herein is a packaging cell line comprising the nucleic acid described in any previous section. In another aspect, provided herein is a packaging cell line comprising the virus described in any previous section. The packaging cell line can be any cell line suitable for packaging Orthopoxvirus viruses (e.g., vaccinia viruses). In certain embodiments, the packaging cell line provided herein is a mammalian packaging cell line (e.g., a human packaging cell line).

Exemplary cells that can be used to culture a virus described herein include, for example, the HeLa cells, U20S cells, 293T cells, NIH3T3 cells, Jurkat cells, 293 cells, COS cells, CHO cells, Saos cells, PC12 cells, and chicken embryo fibroblasts (CEF). Exemplary packaging cell lines that can be used to package a virus described herein include, for example, the HeLa cell line, the U2-OS cell line, the HEK293T cell line, the 786-0 cell line, the A549 cell line or an adherent human cancer cell line. In certain embodiments, the cells also express or are engineered to express one or more factors necessary for the replication and/or packaging of the vaccinia virus.

Also provided herein are methods of propagating a virus described herein using a cell, a cell line, or a packaging cell line as described. In one aspect, provided herein is a method of propagating a virus, comprising culturing a cell, a cell line, or a packaging cell line infected with a virus described herein. In some embodiments, the virus is isolated or purified after propagation.

### C. Exemplary Methods of Genetic Modification

Methods for the insertion or deletion of nucleic acids from a target genome include those described herein and known in the art. Methods for nucleic acid delivery to effect expression of compositions of the present disclosure are believed to include virtually any method by which a nucleic acid (e.g., DNA, including viral and non-viral vectors) can be introduced into an organelle, a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); or by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985). Through the application of techniques such as these, organelle(s), cell(s), tissue(s) or organism(s) may be stably or transiently transformed.

In various embodiments, the Orthopoxviruses are further genetically modified to contain deletions in the B8R gene. The vaccinia virus B8R gene encodes a secreted protein with homology to gamma interferon receptor (IFN-γ). In vitro, the B8R protein binds to and neutralizes the antiviral activity of several species of gamma interferon including human and rat gamma interferon; it does not, however, bind significantly to murine IFN-γ. Deleting the B8R gene prevents the impairment of IFN-γ in humans. In various embodiments, one, two or three trans genes are inserted into the locus of the deleted B8R gene. In some strains, in addition to the transgene(s) present at the site of the B8R deletion, the strain also has, at least one transgene is inserted into an additional locus on the Orthopoxvirus that is not the locus of the deleted B8R gene. In various embodiments, at least one transgene is inserted into boundaries of the 5p deletions, at least one trans gene is inserted into the boundaries of the 3p deletions or both. In various, embodiments at least three, four, five or more trans genes are inserted into the modified Orthopoxvirus genome.

In various embodiments, the modified Orthopoxvirus genome is used as a vector. In some embodiments, the sequence of the modified Orthopoxvirus genome is the sequence depicted below in SEQ ID NO: 15. In some embodiments, the sequence of the modified Orthopoxvirus genome is a derivative of SEQ ID NO: 15. For example, as noted herein, the modified Orthopoxvirus vector may be modified to express one or more transgenes as discussed herein.

In some embodiments, the modified Orthopoxvirus genome was modified by homologous recombination. In some embodiments a 5p targeting construct, which is composed of a transgenic expression cassette flanked on each side by a 1 kb homologous region to C2L and a 1 kb homologous region to F3L, was used for the modification. In some embodiments, the transgenic expression cassette encodes UL40. In some embodiments, the transgenic expression cassette had a sequence set forth in SEQ ID NO: 43. In some embodiments, the transgenic expression cassette encodes K5. In some embodiments, the transgenic expression cassette has a sequence set forth in SEQ ID NO: 44. In some embodiments, the transgenic expression cassette encodes both UL40 and K5. In some embodiments, the transgenic expression cassette has a sequence set forth in SEQ ID NO: 45. In some embodiments, the transgenic expression cassette encodes additional transgenes.

In various embodiments, the modified Orthopoxvirus expresses at least one of the following transgenes: HCMV glycoprotein UL-40 and KSHV K5 protein. Non-limiting examples of sequences of these trans genes and/or of amino acid sequences encoded by them are described in the sequence listing table below.

### IV. PHARMACEUTICAL CARRIER AND COMPOSITIONS

Various embodiments of the invention also provide pharmaceutical compositions comprising a virus or nucleic acid described in any previous section and a physiologically acceptable carrier. In certain embodiments, the pharmaceutical compositions comprise a therapeutically effective amount of the virus. In certain embodiments, the pharmaceutical compositions may be used in one or more methods of treatment described herein including those for treatment of cancer including solid tumors.

Therapeutic compositions containing recombinant Orthopoxvirus vectors of the disclosure can be prepared using methods known in the art. For example, such compositions can be prepared using, e.g., physiologically acceptable carriers, excipients or stabilizers (Remington's Pharmaceutical Sciences 16th edition, Osol, A Ed. (1980); incorporated herein by reference), and in a desired form, e.g., in the form of lyophilized formulations or aqueous solutions.

To induce oncolysis, kill cells, inhibit growth, inhibit metastases, decrease tumor size and otherwise reverse or reduce the malignant phenotype of tumor cells, using the methods and compositions of the present disclosure, one may contact a tumor with the modified Orthopoxvirus, e.g., by administration of the Orthopoxvirus to a patient having cancer by way of, for instance, one or more of the routes of administration described herein. The route of administration may vary with the location and nature of the cancer, and may include, e.g., intradermal, transdermal, parenteral, intravenous, intramuscular, intranasal, subcutaneous, regional (e.g., in the proximity of a tumor, particularly with the vasculature or adjacent vasculature of a tumor), percutaneous, intratracheal, intraperitoneal, intraarterial, intravesical, intratumoral, inhalation, perfusion, lavage, and oral administration and formulation. In specific embodiments, the pharmaceutical composition provided herein is formulated so that it is suitable for the route of administration to be employed. The term “intravascular” is understood to refer to delivery into the vasculature of a patient, meaning into, within, or in a vessel or vessels of the patient. In certain embodiments, the administration is into a vessel considered to be a vein (intravenous), while in others administration is into a vessel considered to be an artery. Veins include, but are not limited to, the internal jugular vein, a peripheral vein, a coronary vein, a hepatic vein, the portal vein, great saphenous vein, the pulmonary vein, superior vena cava, inferior vena cava, a gastric vein, a splenic vein, inferior mesenteric vein, superior mesenteric vein, cephalic vein, and/or femoral vein. Arteries include, but are not limited to, coronary artery, pulmonary artery, brachial artery, internal carotid artery, aortic arch, femoral artery, peripheral artery, and/or ciliary artery. It is contemplated that delivery may be through or to an arteriole or capillary.

Intratumoral injection, or injection directly into the tumor vasculature is specifically contemplated for discrete, solid, accessible tumors. Local, regional or systemic administration also may be appropriate. The viral particles may advantageously be contacted by administering multiple injections to the tumor, spaced, for example, at approximately 1 cm intervals. In the case of surgical intervention, the present disclosure may be used preoperatively, such as to render an inoperable tumor subject to resection. Continuous administration also may be applied where appropriate, for example, by implanting a catheter into a tumor or into tumor vasculature. Such continuous perfusion may take place, for example, for a period of from about 1-2 hours, to about 2-6 hours, to about 6-12 hours, or about 12-24 hours following the initiation of treatment. Generally, the dose of the therapeutic composition via continuous perfusion may be equivalent to that given by a single or multiple injections, adjusted over a period of time during which the perfusion occurs. It is further contemplated that limb perfusion may be used to administer therapeutic compositions of the present disclosure, particularly in the treatment of melanomas and sarcomas.

Treatment regimens may vary, and often depend on tumor type, tumor location, disease progression, and health and age of the patient. Certain types of tumor will require more aggressive treatment, while at the same time, certain patients cannot tolerate more taxing protocols. The clinician will be best suited to make such decisions based on the known efficacy and toxicity (if any) of the therapeutic formulations. In certain embodiments, the tumor being treated may not, at least initially, be resectable. Treatments with the therapeutic agent of the disclosure may increase the resectability of the tumor due to shrinkage at the margins or by elimination of certain particularly invasive portions. Following treatments, resection may be possible. Additional treatments subsequent to resection will serve to eliminate microscopic residual disease at the tumor site.

The treatments may include various “unit doses.” Unit dose is defined as containing a predetermined-quantity of the therapeutic composition. The quantity to be administered, and the particular route and formulation, are within the skill of those in the clinical arts. A unit dose need not be administered as a single injection but may comprise continuous infusion over a set period of time. Unit dose of the present disclosure may conveniently be described in terms of plaque forming units (pfu) for a viral construct. Unit doses may range from 103, 104, 105, 106, 107, 108, 109, 1010, 1011, 1012, to 1013 pfu and higher. Additionally or alternatively, depending on the kind of virus and the titer attainable, one may deliver 1 to 100, 10 to 50, 100-1000, or up to about or at least about 1×104, 1×105, 1×106, 1×107, 1×108, 1×109, 1×1010, 1×1011, 1×1012, 1×1013, 1×1014, or 1×1015 or higher infectious viral particles (vp), including all values and ranges there between, to the tumor or tumor site.

Another method of delivery of the recombinant Orthopoxvirus genome disclosed herein to cancer or tumor cells may be via intratumoral injection. However, the pharmaceutical compositions disclosed herein may alternatively be administered parenterally, intravenously, intradermally, intramuscularly, transdermally or even intraperitoneally as described in U.S. Pat. Nos. 5,543,158; 5,641,515 and 5,399,363 (each specifically incorporated herein by reference in its entirety). Injection of nucleic acid constructs may be delivered by syringe or any other method used for injection of a solution, as long as the expression construct can pass through the particular gauge of needle required for injection. An exemplary needleless injection system that may be used for the administration of recombinant Orthopoxviruses described herein is exemplified in U.S. Pat. No. 5,846,233. This system features a nozzle defining an ampule chamber for holding the solution and an energy device for pushing the solution out of the nozzle to the site of delivery. Another exemplary syringe system is one that permits multiple injections of predetermined quantities of a solution precisely at any depth (U.S. Pat. No. 5,846,225).

Mixtures of the viral particles or nucleic acids described herein may be prepared in water suitably mixed with one or more excipients, carriers, or diluents. Dispersions may also be prepared in glycerol, liquid polyethylene glycols, and mixtures thereof and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form may be sterile and may be fluid to the extent that easy syringability exists. It may be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution may be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety, and purity standards as required by FDA Office of Biologics standards.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. The phrase “pharmaceutically acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

### V. KITS

The nucleic acids, viruses, pharmaceutical carriers and compositions provided herein can be packaged as kits. Kits can optionally include one or more of the following: instructions for use, devices, reagents, and components, such as tubes, containers or syringes. Exemplary kits can include any nucleic acid or virus provided herein and can optionally include instructions for use, a device for administering the virus or nucleic acid to a subject.

In one aspect, provided herein is a kit comprising a nucleic acid described in sections II and III and a package insert instructing a user of the kit to express the nucleic acid in a host cell. In some embodiments, the kit includes a host cell line. In some embodiments, the kit includes devices to detect viral propagation in a host cell line. In some embodiments, the kit includes devices to detect gene expression, such as a device to detect red or green fluorescent protein which may be encoded in the nucleic acid.

In another aspect, provided herein is a kit comprising a virus described in section II and III and a package insert instructing a user to administer a therapeutically effective amount of the virus to a subject (e.g., a mammalian subject, such as a human subject) having cancer, thereby treating the cancer. In certain embodiments, the mammalian subject is a human subject. The cancer to be treated can be a cancer described in VI.

In another aspect, provided herein is a kit comprising a virus or nucleic acid described in section II and III, a second therapeutic agent for the treatment of cancer, and optionally a package insert instructing a user to administer a therapeutically effective amount of the virus and the second therapeutic agent to a subject (e.g., a mammalian subject, such as a human subject) having cancer, thereby treating the cancer. In certain embodiments, the mammalian subject is a human subject. The cancer to be treated can be a cancer described in VI. In some embodiments, the kit includes host cells and/or other reagents for generating a virus from the nucleic acid. In some embodiments, the instructions designate the second therapeutic agent is to be administered before, concurrently with or after administering the recombinant Orthopoxvirus. In some embodiments, the second therapeutic agent is a CAR. In some embodiments, the second therapeutic agent is a checkpoint blockade immunotherapy. In some embodiments, the checkpoint blockade immunotherapy is a PD1 and/or a PD-L1 inhibitor. In some embodiments, the second therapeutic agent is an immunomodulatory agent. In some embodiments, the immunomodulatory agent is a CD47. In some embodiments, the immunomodulatory agent is an NKG2A inhibitor.

In another aspect, provided herein is a kit comprising a pharmaceutical composition as described in section IV that contains a recombinant Orthopoxvirus. In some embodiments, the kit includes a package insert instructing a user to administer a therapeutically effective amount of the pharmaceutical composition to a subject (e.g., a mammalian subject, such as a human subject) having cancer, thereby treating the cancer. In certain embodiments, the mammalian subject is a human subject. The cancer to be treated can be a cancer described in VI. In some embodiments, the kit includes a second therapeutic agent for the treatment of cancer. In some embodiments, the instructions designate the second therapeutic agent is to be administered before, concurrently with or after administering the pharmaceutical composition. In some embodiments, the second therapeutic agent is a CAR. In some embodiments, the second therapeutic agent is a checkpoint blockade immunotherapy. In some embodiments, the checkpoint blockade immunotherapy is a PD1 and/or a PD-L1 inhibitor. In some embodiments, the second therapeutic agent is an immunomodulatory agent. In some embodiments, the immunomodulatory agent is a CD47. In some embodiments, the immunomodulatory agent is an NKG2A inhibitor.

In some embodiments, the kit includes a nucleic acid, a virus, or a pharmaceutical composition described in any previous section along with devices, instructions, and or reagents for the user to produce a second therapeutic agent. In some embodiments, the kit includes a package insert instructing a user to administer a therapeutically effective amount of the pharmaceutical composition to a subject (e.g., a mammalian subject, such as a human subject) having cancer, thereby treating the cancer. In some embodiments the second therapeutic agent is an autologous tumor lymphocyte (TIL) therapy. In some embodiments, the Orthopoxvirus or pharmaceutical composition is administered to the subject prior to harvesting the TILs from a tumor from the subject for producing the autologous TIL therapy.

In preferred embodiments, the nucleic acid or the virus is stored in one or more containers suitable for storing the nucleic acid or the virus. In certain embodiments, the kits provided herein further comprise controls suitable for their intended use.

### VI. METHODS OF TREATMENT AND COMBINATION THERAPY

Also provided herein are methods of treating a cell proliferation disorder, such as cancer in a subject (e.g., a mammalian subject, such as a human subject).

In one aspect, provided herein is a method of treating a cell proliferation disorder, such as cancer in a subject (e.g., a mammalian subject, such as a human subject), the method comprising administering to the subject (e.g., a mammalian subject, such as a human subject) a therapeutically effective amount of a virus described in section III.

In another aspect, provided herein is a method of treating a cell proliferation disorder, such as cancer in a subject (e.g., a mammalian subject, such as a human subject), the method comprising administering to the subject (e.g., a mammalian subject, such as a human subject) a therapeutically effective amount of a pharmaceutical composition described in section IV.

In a specific embodiment of the method of treating described herein, the mammalian subject is a human subject.

In certain embodiments of the method of treating described herein, the cancer is selected from the group consisting of leukemia, lymphoma, liver cancer, bone cancer, lung cancer, brain cancer, bladder cancer, gastrointestinal cancer, breast cancer, cardiac cancer, cervical cancer, uterine cancer, head and neck cancer, gallbladder cancer, laryngeal cancer, lip and oral cavity cancer, ocular cancer, melanoma, pancreatic cancer, prostate cancer, colorectal cancer, testicular cancer, and throat cancer.

In certain embodiments of the method of treating described herein, the cancer is selected from the group consisting of acute lymphoblastic leukemia (ALL), acute myeloid leukemia (AML), chronic lymphocytic leukemia (CLL), chronic myelogenous leukemia (CML), adrenocortical carcinoma, AIDS-related lymphoma, primary CNS lymphoma, anal cancer, appendix cancer, astrocytoma, atypical teratoid/rhabdoid tumor, basal cell carcinoma, bile duct cancer, extrahepatic cancer, Ewing sarcoma family, osteosarcoma and malignant fibrous histiocytoma, central nervous system embryonal tumors, central nervous system germ cell tumors, craniopharyngioma, ependymoma, bronchial tumors, Burkitt lymphoma, carcinoid tumor, primary lymphoma, chordoma, chronic myeloproliferative neoplasms, colon cancer, extrahepatic bile duct cancer, ductal carcinoma in situ (DCIS), endometrial cancer, ependymoma, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors (GIST), testicular germ cell tumor, gestational trophoblastic disease, glioma, childhood brain stem glioma, hairy cell leukemia, hepatocellular cancer, Langerhans cell histiocytosis, Hodgkin lymphoma, hypopharyngeal cancer, islet cell tumors, pancreatic neuroendocrine tumors, Wilms tumor and other childhood kidney tumors, Langerhans cell histiocytosis, small cell lung cancer, cutaneous T cell lymphoma, intraocular melanoma, Merkel cell carcinoma, mesothelioma, metastatic squamous neck cancer, midline tract carcinoma, multiple endocrine neoplasia syndromes, multiple myeloma/plasma cell neoplasm, myelodysplastic syndromes, nasal cavity and paranasal sinus cancer, nasopharyngeal cancer, neuroblastoma, non-Hodgkin's lymphoma (NHL), non-small cell lung cancer (NSCLC), epithelial ovarian cancer, germ cell ovarian cancer, low malignant potential ovarian cancer, pancreatic neuroendocrine tumors, papillomatosis, paraganglioma, paranasal sinus and nasal cavity cancer, parathyroid cancer, penile cancer, pharyngeal cancer, pheochromocytoma, pituitary tumor, pleuropulmonary blastoma, primary peritoneal cancer, rectal cancer, retinoblastoma, rhabdomyosarcoma, salivary gland cancer, Kaposi's sarcoma, rhabdomyosarcoma, Sezary syndrome, small intestine cancer, soft tissue sarcoma, throat cancer, thymoma and thymic carcinoma, thyroid cancer, transitional cell cancer of the renal pelvis and ureter, urethral cancer, endometrial uterine cancer, uterine sarcoma, vaginal cancer, vulvar cancer, and Waldenstrom macroglobulinemia.

In some embodiments, the cancer can be a melanoma, ovarian cancer, cervical cancer, lung cancer, bladder cancer, breast cancer, head and neck cancer, renal cell carcinoma, acute myeloid leukemia, colorectal cancer, and sarcoma. In some embodiments, the cancer is a cancer with a high mutational burden. In some embodiments, the cancer is melanoma, lung squamous, lung adenocarcinoma, bladder cancer, lung small cell cancer, esophageal cancer, colorectal cancer, cervical cancer, head and neck cancer, stomach cancer or uterine cancer.

In some embodiments, the cancer is an epithelial cancer. In some embodiments, the cancer is selected from non-small cell lung cancer (NSCLC), CRC, ovarian cancer, breast cancer, esophageal cancer, gastric cancer, pancreatic cancer, cholangiocarcinoma cancer, endometrial cancer. In some embodiments, the breast cancer is HR+/Her2− breast cancer. In some embodiments, the breast cancer is a triple negative breast cancer (TNBC). In some embodiments, the breast cancer is a HER2+ breast cancer.

In some embodiments, the subject has a cancer that is a hematological tumor. Non-limiting examples of hematological tumors include leukemia, including acute leukemias (such as 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

In some embodiments, the subject has a solid tumor cancer. Non-limiting examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyngioma, ependymoma, pinealoma, hemangioblastoma, acoustic neuroma, oligodendroglioma, meningioma, melanoma, neuroblastoma and retinoblastoma). In several examples, a tumor is melanoma, lung cancer, lymphoma breast cancer or colon cancer.

In some embodiments, the cancer is a skin cancer. In particular embodiments, the cancer is a melanoma, such as a cutaneous melanoma. In some embodiments, the cancer is a merkel cell or metastatic cutaneous squamous cell carcinoma (CSCC).

In some embodiments, the tumor is a carcinoma, which is a cancer that develops from epithelial cells or is a cancer of epithelial origin. In some embodiments, the cancer arises from epithelial cells which include, but are not limited to, breast cancer, basal cell carcinoma, adenocarcinoma, gastrointestinal cancer, lip cancer, mouth cancer, esophageal cancer, small bowel cancer and stomach cancer, colon cancer, liver cancer, bladder cancer, pancreas cancer, ovary cancer, cervical cancer, lung cancer, breast cancer and skin cancer, such as squamous cell and basal cell cancers, prostate cancer, renal cell carcinoma, and other known cancers that effect epithelial cells throughout the body.

In some embodiments, the subject has a cancer that is a gastrointestinal cancer involving a cancer of the gastrointestinal tract (GI tract), including cancers or the upper or lower digestive tract, or an accessory organ of digestion, such as esophagus, stomach, biliary system, pancreas, small intestine, large intestine, rectum or anus. In some embodiments, the cancer is an esophageal cancer, stomach (gastric) cancer, pancreatic cancer, liver cancer (hepatocellular carcinoma), gallbladder cancer, cancer of the mucosa-associated lymphoid tissue (MALT lymphoma), cancer of the biliary tree, colorectal cancer (including colon cancer, rectum cancer or both), anal cancer, or a gastrointestinal carcinoid tumor. In particular embodiments, the cancer is a colorectal cancer.

In some embodiments, the cancer is a colorectal cancer. Colorectal cancer (CRC) is a common tumor of increasing incidence, which, in many cases, does not response to checkpoint inhibition or other immunotherapy. This is the case even though such cancers have properties that are associated with response, e.g. a reasonably high mutation rate and well established association of prognosis with level of T cell infiltration.

In some embodiments, the cancer is an ovarian cancer. In some embodiments, the cancer is a triple-negative breast cancer (TNBC).

In some embodiments, the cancer is lung cancer. In some embodiments, the cancer is a breast cancer. In some embodiments, the cancer is a colorectal cancer. In some embodiments, the cancer is pancreatic cancer. In some embodiments, the cancer is a merkel cell cancer. In some embodiments, the cancer is a metastatic cutaneous squamous cell carcinoma (CSCC). In some embodiments, the cancer is a melanoma.

In some embodiments, the subject is one whose cancer is refractory to, and or who has relapsed following treatment with, a checkpoint blockade, such as an anti-PD1 or anti-PD-L1 therapy.

In some embodiments of the method of treating described herein, the virus and the pharmaceutical composition are not administered in combination with another agent for treating the cell proliferation disorder (such as cancer).

In other embodiments of the method of treating described herein, the virus or the pharmaceutical composition is administered in combination with one or more additional agents or transgenes for treating the cell proliferation disorder (such as cancer), for example, the one or more additional agents or transgenes described in sections III and VI.

In various embodiments, the recombinant Orthopoxvirus and the pharmaceutical composition disclosed herein can be administered to a subject, e.g., a mammalian subject, such as a human, suffering from a cell proliferation disorder, such as cancer, e.g., to kill cancer cells directly by oncolysis and/or to enhance the effectiveness of the adaptive immune response against the target cancer cells. In some embodiments, the cell proliferation disorder is a cancer, such as leukemia, lymphoma, liver cancer, bone cancer, lung cancer, brain cancer, bladder cancer, gastrointestinal cancer, breast cancer, cardiac cancer, cervical cancer, uterine cancer, head and neck cancer, gallbladder cancer, laryngeal cancer, lip and oral cavity cancer, ocular cancer, melanoma, pancreatic cancer, prostate cancer, colorectal cancer, testicular cancer, or throat cancer. In particular cases, the cell proliferation disorder may be a cancer selected from the group consisting of acute lymphoblastic leukemia (ALL), acute myeloid leukemia (AML), chronic lymphocytic leukemia (CLL), chronic myelogenous leukemia (CML), adrenocortical carcinoma, AIDS-related lymphoma, primary CNS lymphoma, anal cancer, appendix cancer, astrocytoma, atypical teratoid/rhabdoid tumor, basal cell carcinoma, bile duct cancer, extrahepatic cancer, ewing sarcoma family, osteosarcoma and malignant fibrous histiocytoma, central nervous system embryonal tumors, central nervous system germ cell tumors, craniopharyngioma, ependymoma, bronchial tumors, burkitt lymphoma, carcinoid tumor, primary lymphoma, chordoma, chronic myeloproliferative neoplasms, colon cancer, extrahepatic bile duct cancer, ductal carcinoma in situ (DCIS), endometrial cancer, ependymoma, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors (GIST), testicular germ cell tumor, gestational trophoblastic disease, glioma, childhood brain stem glioma, hairy cell leukemia, hepatocellular cancer, langerhans cell histiocytosis, hodgkin lymphoma, hypopharyngeal cancer, islet cell tumors, pancreatic neuroendocrine tumors, wilms tumor and other childhood kidney tumors, langerhans cell histiocytosis, small cell lung cancer, cutaneous T-cell lymphoma, intraocular melanoma, merkel cell carcinoma, mesothelioma, metastatic squamous neck cancer, midline tract carcinoma, multiple endocrine neoplasia syndromes, multiple myeloma/plasma cell neoplasm, myelodysplastic syndromes, nasal cavity and paranasal sinus cancer, nasopharyngeal cancer, neuroblastoma, non-hodgkin lymphoma (NHL), non-small cell lung cancer (NSCLC), epithelial ovarian cancer, germ cell ovarian cancer, low malignant potential ovarian cancer, pancreatic neuroendocrine tumors, papillomatosis, paraganglioma, paranasal sinus and nasal cavity cancer, parathyroid cancer, penile cancer, pharyngeal cancer, pheochromocytoma, pituitary tumor, pleuropulmonary blastoma, primary peritoneal cancer, rectal cancer, retinoblastoma, rhabdomyosarcoma, salivary gland cancer, kaposi sarcoma, rhabdomyosarcoma, sezary syndrome, small intestine cancer, soft tissue sarcoma, throat cancer, thymoma and thymic carcinoma, thyroid cancer, transitional cell cancer of the renal pelvis and ureter, urethral cancer, endometrial uterine cancer, uterine sarcoma, vaginal cancer, vulvar cancer, and Waldenstrom macroglobulinemia.

A physician or other medical professional having ordinary skill in the art can readily determine an effective amount of the recombinant Orthopoxvirus vector for administration to a subject, e.g., a mammalian subject (e.g., a human) in need thereof. For example, a physician may start prescribing doses of recombinant Orthopoxvirus vector at levels lower than that required in order to achieve the desired therapeutic effect and gradually increase the dosage until the desired effect is achieved. Alternatively, a physician may begin a treatment regimen by administering a dose of recombinant Orthopoxvirus vector and subsequently administer progressively lower doses until a therapeutic effect is achieved (e.g., a reduction in the volume of one or more tumors). In general, a suitable daily dose of a recombinant Orthopoxvirus vector of the disclosure will be an amount of the recombinant Orthopoxvirus vector which is the lowest dose effective to produce a therapeutic effect. A daily dose of a therapeutic composition of the recombinant Orthopoxvirus vector of the disclosure may be administered as a single dose or as two, three, four, five, six or more doses administered separately at appropriate intervals throughout the day, week, month, or year, optionally, in unit dosage forms. While it is possible for the recombinant Orthopoxvirus vector of the disclosure to be administered alone, it may also be administered as a pharmaceutical formulation in combination with excipients, carriers, and optionally, additional therapeutic agents.

Recombinant Orthopoxvirus vectors of the disclosure can be monitored for their ability to attenuate the progression of a cell proliferation disease, such as cancer, by any of a variety of methods known in the art. For instance, a physician may monitor the response of a subject, e.g., a mammalian subject (e.g., a human) to treatment with recombinant Orthopoxvirus vector of the disclosure by analyzing the volume of one or more tumors in the subject. Alternatively, a physician may monitor the responsiveness of a subject (e.g., a human) t to treatment with recombinant Orthopoxvirus vector of the disclosure by analyzing the T-reg cell population in the lymph of a particular subject. For instance, a physician may withdraw a sample from a subject, e.g., a mammalian subject (e.g., a human) and determine the quantity or density of cancer cells using established procedures, such as fluorescence activated cell sorting. A finding that the quantity of cancer cells in the sample has decreased (e.g., by 1%, 2%, 3%, 4%, 5%, 6%, 7%, 8%, 9%, 10%, 20%, 30%, 40%, 50%, 60%, 70%, 80%, 90%, 95%, 96%, 97%, 98%, 99%, or more) relative to the quantity of cancer cells in a sample obtained from the subject prior to administration of the recombinant Orthopoxvirus may be an indication that the Orthopoxvirus administration is effectively treating the cancer.

### A. Combination Therapy

In various embodiments, the recombinant Orthopoxvirus vectors or pharmaceutical compositions described herein may be administered with one or more additional agents, such as an immune checkpoint inhibitor. For instance, the recombinant Orthopoxvirus vector can be administered simultaneously with, admixed with, or administered separately from an immune checkpoint inhibitor. Exemplary immune checkpoint inhibitors for use in conjunction with the compositions and methods of the disclosure include but are not limited to OX40 ligand, ICOS ligand, anti-CD47 antibody or antigen-binding fragment thereof, anti-CD40/CD40L antibody or antigen-binding fragment thereof, anti-Lag3 antibody or antigen-binding fragment thereof, anti-CTLA-4 antibody or antigen-binding fragment thereof, anti-PD-LI antibody or antigen-binding fragment thereof, anti-PDl antibody or antigen-binding fragment thereof, and anti-Tim-3 antibody or antigen-binding fragment thereof. Additionally or alternatively, a vector of the disclosure can be administered simultaneously with, admixed with, or administered separately from an interleukin (IL). For instance, the recombinant Orthopoxvirus vector can be administered simultaneously with, admixed with, or administered separately from an interleukin. Exemplary interleukins for use in conjunction with the compositions and methods of the disclosure include but are not limited to IL-1 alpha, IL-1 beta, IL-2, IL-4, IL-7, IL-10, IL-12 p35, IL-12 p40, IL-12 p70, IL-15, IL-18, IL-21, and IL-23. Additionally or alternatively, a vector of the disclosure can be administered simultaneously with, admixed with, or administered separately from an interferon. For instance, the recombinant Orthopoxvirus vector can be administered simultaneously with, admixed with, or administered separately from an interferon.

Exemplary interferons for use in conjunction with the compositions and methods of the disclosure include but are not limited to IFN-alpha, IFN-beta, IFN-delta, IFN-epsilon, IFN-tau, IFN-omega, IFN-zeta, and IFN-gamma. Additionally or alternatively, a vector of the disclosure can be administered simultaneously with, admixed with, or administered separately from a TNF superfamily member protein. For instance, the recombinant Orthopoxvirus vector can be administered simultaneously with, admixed with, or administered separately from a TNF superfamily member protein. Exemplary TNF superfamily member proteins for use in conjunction with the compositions and methods of the disclosure include but are not limited to TRAIL, Fas ligand, LIGHT (TNFSF-14), TNF-alpha, and 4-1BB ligand. Additionally or alternatively, a vector of the disclosure can be administered simultaneously with, admixed with, or administered separately from a cytokine. For instance, the recombinant Orthopoxvirus vector can be administered simultaneously with, admixed with, or administered separately from a cytokine. Exemplary cytokines for use in conjunction with the compositions and methods of the disclosure includes but are not limited to GM-CSF, Flt3 ligand, CD40 ligand, anti-TGF-beta, anti-VEGF-R2, and cGAS (guanyl adenylate cyclase).

Additionally or alternatively, immune checkpoint inhibitors may be expressed in the Orthopoxvirus itself. For instance, the recombinant Orthopoxvirus vector can include a transgene encoding an immune checkpoint inhibitor. Exemplary immune checkpoint inhibitors for expression by the Orthopoxvirus of the compositions and methods of the disclosure include but are not limited to OX40 ligand, ICOS ligand, anti-CD47 antibody or antigen-binding fragment thereof, anti-CD40/CD40L antibody or antigen-binding fragment thereof, anti-Lag3 antibody or antigen-binding fragment thereof, anti-CTLA-4 antibody or antigen-binding fragment thereof, anti-PD-LI antibody or antigen-binding fragment thereof, anti-PDl antibody or antigen-binding fragment thereof, and anti-Tim-3 antibody or antigen-binding fragment thereof. Additionally or alternatively, interleukins may be expressed in the Orthopoxvirus itself. For instance, the recombinant Orthopoxvirus vector can include a transgene encoding an interleukin. Exemplary immune checkpoint inhibitors for expression by the Orthopoxvirus of the compositions and methods of the disclosure include but are not limited to IL-1 alpha, IL-1 beta, IL-2, IL-4, IL-7, IL-10, IL-12 p35, IL-12 p40, IL-12 p70, IL-15, IL-18, IL-21, and IL-23. Additionally or alternatively, interferons may be expressed in the Orthopoxvirus itself. For instance, the recombinant Orthopoxvirus vector can include a transgene encoding an interferon. Exemplary interferons for expression by the Orthopoxvirus of the compositions and methods of the disclosure include but are not limited to IFN-alpha, IFN-beta, IFN-delta, IFN-epsilon, IFN-tau, IFN-omega, IFN-zeta, and IFN-gamma. Additionally or alternatively, TNF superfamily member proteins may be expressed in the Orthopoxvirus itself. For instance, the recombinant Orthopoxvirus vector can include a transgene encoding a TNF superfamily member protein. Exemplary TNF superfamily member proteins for expression by the Orthopoxvirus of the compositions and methods of the disclosure include but are not limited to TRAIL, Fas ligand, LIGHT (TNFSF-I4), TNF-alpha, and 4-IBB ligand. Additionally or alternatively, cytokines may be expressed in the Orthopoxvirus itself. For instance, the recombinant Orthopoxvirus vector can include a transgene encoding a cytokine. Exemplary cytokines for expression by the Orthopoxvirus of the compositions and methods of the disclosure include but are not limited to GM-CSF, fins-related tyrosine kinase 3 (Flt3) ligand, CD40 ligand, TGF-beta, VEGF-R2, and c-KIT.

Additionally or alternatively, tumor-associated antigens may be expressed in the Orthopoxvirus itself. For instance, the recombinant Orthopoxvirus vector can include a transgene encoding a tumor-associated antigen. Exemplary tumor-associated antigens for expression by the Orthopoxvirus of the compositions and methods of the disclosure include but are not limited to CDI9, CD33, EpCAM, CEA, PSMA, EGFRvIII, CD133, EGFR, CDHI9, ENPP3, DLL3, MSLN, RORI, HER2, HLAA2, EpHA2, EpHA3, MCSP, CSPG4, NG2, RON, FLT3, BCMA, CD20, FAPα, FRα, CA-9, PDGFRα, PDGFRβ, FSPI, S100A4, ADAMI2m, RET, MET, FGFR, INSR, NTRK, MAGE-A3, NY-ESO-I, one or more human papillomavirus (HPV) proteins, E6 and E7 proteins of HPVI6, E6 and E7 proteins of HPVI8, brachyury, or prostatic acid phosphatase, or one or more fragments thereof.

In certain embodiments of the method of treating described herein, the method further comprises administering to the subject (e.g., a mammalian subject, such as a human subject) an immune checkpoint inhibitor. In specific embodiments, the immune checkpoint inhibitor is selected from the group consisting of OX40 ligand, ICOS ligand, anti-CD47 antibody or antigen-binding fragment thereof, anti-CD40/CD40L antibody or antigen-binding fragment thereof, anti-Lag3 antibody or antigen-binding fragment thereof, anti-CTLA-4 antibody or antigen-binding fragment thereof, anti-PD-LI antibody or antigen-binding fragment thereof, anti-PD I antibody or antigen-binding fragment thereof, and anti-Tim-3 antibody or antigen-binding fragment thereof. In a specific embodiment, the immune checkpoint inhibitor is an anti-PD I antibody or antigen-binding fragment thereof or an anti-CTLA-4 antibody or antigen-binding fragment thereof. In another specific embodiment, the immune checkpoint inhibitor is an anti-PD I antibody or antigen-binding fragment thereof. In another specific embodiment, the immune checkpoint inhibitor is an anti-CTLA-4 antibody or antigen-binding fragment thereof. In another specific embodiment, the immune checkpoint inhibitor is an anti-PD-LI antibody or antigen-binding fragment thereof. In a specific embodiment, the immune checkpoint inhibitor is ipilimumab. In another specific embodiment, the immune checkpoint inhibitor is tremelimumab. In another specific embodiment, the immune checkpoint inhibitor is nivolumab. In another specific embodiment, the immune checkpoint inhibitor is pembrolizumab. In another specific embodiment, the immune checkpoint inhibitor is cemiplimab. In another specific embodiment, the immune checkpoint inhibitor is atezolizumab. In another specific embodiment, the immune checkpoint inhibitor is avelumab. In another specific embodiment, the immune checkpoint inhibitor is durvalumab.

In certain embodiments of the method of treating described herein, the method further comprises administering to the subject (e.g., a mammalian subject, such as a human subject) an interleukin. In specific embodiments, the interleukin is selected from the group consisting of IL-1 alpha, IL-1 beta, IL-2, IL-4, IL-7, IL-10, IL-12 p35, IL-12 p40, IL-12 p70, IL-15, IL-18, IL-21, and IL-23. In specific embodiments, the interleukin is selected from the group consisting of IL-12 p35, IL-12 p40, and IL-12 p70. In specific embodiments, the interleukin is membrane-bound.

In certain embodiments of the method of treating described herein, the method further comprises administering to the subject (e.g., a mammalian subject, such as a human subject) an interferon. In specific embodiments, the interferon is selected from the group consisting of IFN-alpha, IFN-beta, IFN-delta, IFN-epsilon, IFN-tau, IFN-omega, IFN-zeta, and IFN-gamma.

In certain embodiments of the method of treating described herein, the method further comprises administering to the subject (e.g., a mammalian subject, such as a human subject) a cytokine. In specific embodiments, the cytokine is a TNF superfamily member protein. In a specific embodiment, the TNF superfamily member protein is selected from the group consisting of TRAIL, Fas ligand, LIGHT (TNFSF-14), TNF-alpha, and 4-1BB ligand. In specific embodiments, the cytokine is selected from the group consisting of GM-CSF, Flt3 ligand, CD40 ligand, TGF-beta, VEGF-R2, and cKit. In a specific embodiment, the cytokine is Flt3 ligand.

B. Combination Therapy with TILs

Various embodiments of the invention provide methods and uses involving combination therapies involving a T lymphocyte infiltrating (TIL) cell therapy and an oncolytic virus (OV). Also among embodiments provided herein are methods and uses involving a TIL cell therapy in a subject who has previously received treatment with an oncolytic virus. Further among embodiments provided herein are methods and uses involving an oncolytic cell therapy in a subject (also referred to as a patient) who has previously received treatments with a TIL cell therapy. In some embodiments, the methods can be used for treating subjects having or suspected of having a cancer, such as a cancerous solid tumor also referred to as a solid tumor.

Embodiments of such methods and uses include therapeutic methods and uses, for example, involving administration of the therapeutic cells, or compositions containing the same, to a subject having a cancer. In some cases, the cancer is a tumor. In some embodiments, the cancer is a solid tumor and the methods and uses are for treating a solid tumor in the subject. In some embodiments, the oncolytic virus and cells, or pharmaceutical compositions thereof, are administered in an effective amount to effect treatment of the cancer. Uses include uses of the TIL cell therapy and oncolytic virus, or pharmaceutical compositions thereof, in such methods and treatments, and in the preparation of one or more medicaments in order to carry out such therapeutic methods. In some embodiments, the methods thereby treat the cancer in the subject.

In some embodiments, the TIL cell therapy includes tumor-reactive T cells though other cells are contemplated as well. In some embodiments, the tumor reactive T cells are obtained from a subject (e.g. a subject with the cancer to be treated) and are enriched ex vivo by processes of selection and expansion. In some embodiments, the tumor-reactive T cells are T cells that recognize a cancer neoantigen. The majority of neoantigens arise from passenger mutations, meaning they do not infer any growth advantage to the cancer cell. A smaller number of mutations actively promote tumor growth, these are known as driver mutations. Passenger mutations are likely to give rise to neoantigens that are unique to each patient and may be present in a subset of all cancer cells. Driver mutations give rise to neoantigens that are likely to be present in all the tumor cells of an individual and potentially shared. In some embodiments of the provided method, the population of T cells include tumor-reactive T cells that can recognize neoantigens containing passenger and/or driver mutations.

In particular aspects, the provided methods can be used for the ex vivo production of a T cell therapy, including in various embodiments for the ex vivo expansion of autologous tumor-reactive T cells. In some aspects, neoantigens are ideal targets for immunotherapies because they represent disease-specific targets. For example, such antigens generally are not present in the body before the cancer developed and are truly cancer specific, not expressed on normal cells and are not subjected to off target immune toxicity. Thus, the unique repertoire of neoantigens specific to the patient can elicit a strong immune response specific to the cancer cells, avoiding normal cells. This is an advantage over other cell therapy targets that may not be disease-specific targets, since even low levels of target antigen on normal cells can lead to severe fatal autoimmune toxicity in the context of engineered therapies that target common antigens. For example, an anti MAGE-A3-TCR program in melanoma patients was halted due to study related deaths attributed to cross reactivity with a similar target MAGE-A12, which is expressed at a low level in the brain. A significant challenge in cancer immunotherapy has been the identification of cancer targets.

Recent clinical studies have demonstrated that T cells isolated from surgically resected tumors possess TCRs that recognize neoantigens, and expanding these neoantigen reactive TIL populations and re-infusing them into the patient can in some cases result in a dramatic clinical benefit. This personalized therapy has generated remarkable clinical responses in certain patients with common epithelial tumors.

Existing methods for obtaining and generating tumor-reactive T cells are not entirely satisfactory. For example, direct isolation of tumor-reactive T cells from a subject without expansion is not feasible because therapeutically effective amounts of such cells cannot be obtained. As an alternative, attempts have been made to identify TCRs specific to a desired neoantigen for recombinant engineering of the TCR into T cells for use in adoptive cell therapy methods. Such approaches, however, produce only a single TCR against a specific neoantigen and thereby lack diversity to recognize a broader repertoire of multiple tumor-specific mutations. Other methods involve bulk expansion of T cells from a tumor source, which has the risk of expanding T cells that are not reactive to a tumor antigen and/or that may include a number of bystander cells that could exhibit inhibitory activity. For example, tumor regulatory T cells (Tregs) are a subpopulation of CD4⁺ T cells, which specialize in suppressing immune responses and could limit reactivity of a T cell product. These further approaches that have sought to expand tumor-reactive T cells ex vivo are not selective such that non-reactive T cells in the culture may preferentially expand over reactive T cells resulting in a final product that lacks satisfactory reactivity and/or in which the number of tumor-reactive T cells remains insufficient. Methods to produce tumor-reactive T cells for therapy are needed.

In some embodiments, the TIL cell therapy includes T cells from a tumor that have been enriched and expanded ex vivo. In some embodiments, the T cells are enriched based on selection of upregulation (or activation) markers that become upregulated on tumor reactive cells after presentation of neoantigens, followed by expansion of T cells enriched for tumor-reactive T cells. In some cases, the methods may also involve co-culture with antigen presenting cells presenting peptide neoepitopes. For example, the methods of culturing the cells include methods to proliferate and expand cells, particularly involving steps to enrich for proliferation and expansion of tumor-reactive T cells such as by selection of such cells, or based on certain selection markers that are associated with or indicative of tumor-reactive T cells.

The TIL therapy for use in the provided combination therapy methods and uses include any as described in PCT publication No. WO2020/172202, WO2020/205662, WO2021/108727, and WO2021/174208, each incorporated by reference in their entirety.

In some embodiments, the TILs are produced by a process in which a population containing T cells is obtained, selected or isolated from a tumor sample from a subject, such as a human subject. In some embodiments, the tumor sample is a tumor fragment or suspension of cell therefrom containing tumor infiltrating lymphocytes or TILs. In some embodiments, the provided methods include (1) Enriching a population containing T cells obtained from a tumor sample of a donor subject to produce a first population of T cells; (2) Stimulating the first population with one or more T-cell stimulating agents of lymphocytes to produce a second population of activated T cells; (3) Co-culturing cells from the second population of T cells in the presence of antigen presenting cells that present one or more peptide (e.g. peptide neoepitopes) on an MHC (MHC-associated non-native peptide), in which the co-culturing produces a third population of cells containing or enriched for T cells that are reactive to a peptides presented on the MHC of an APC (e.g. tumor reactive T cells); (4) from the third population, separating the APCs to produce a fourth population of T cells containing endogenous TCR that are reactive to peptides present on the APCs; and (5) expanding the fourth population of T cells containing tumor reactive T cells by incubation in the presence of T cell stimulating agents. In some embodiments, the separating of the T cells in (4) can involve depleting or removing the APCs and/or can include selection of T cells based on upregulation markers on reactive or activated T cells. In some embodiments, one or more of the steps can be carried out in a closed system using serum free medium.

In the provided methods, tumor reactive T cells are identified or enriched from the stimulated T cells expanded in the first step by one or more further steps that include ex vivo co-culture of the stimulated T cells (second population of T cells) with antigen presenting cells (APCs) and one or a plurality of peptides that include neoepitopes of a tumor antigen (APCs/peptide neoepitopes). In some embodiments, provided methods include ex vivo co-culture in which the second population of T cells are incubated with APCs, such as autologous APCs or artificial antigen presenting cells (aAPCs), that have been exposed to or contacted with one or more peptides, e.g. synthetic peptides, under conditions in which the APCs have been induced to present one or more peptides from a tumor-associated antigen. In some embodiments, the population of T cells are autologous T cells from a subject with a tumor and the source of synthetic peptides are tumor antigenic peptides from a tumor antigen of the same subject. In some embodiments, cells from the ex vivo co-culture are a population of cells (third population) that include tumor reactive T cells that recognize or are activated by a peptide presented on an MHC of an APC in the culture. In some embodiments, cells from the ex vivo co-culture represent a source of cells that are enriched for tumor reactive T cells.

In some cases, the tumor reactive T cells can be further enriched by separation or selection of cells that express one or more upregulation marker, such as an activation marker, associated with tumor-reactive T cells (the further separation or selection producing a fourth population of T cells of the enriched tumor reactive T cells). The T cell activation markers can include cell surface markers whose expression is upregulated or specific to T cells that have been exposed to antigen and activated. Exemplary of such markers are described below.

In particular embodiments, the provided methods include enriching from a biological sample (directly sourced from a sample in vivo or from an ex vivo coculture with antigen presenting cells (APCs)) T cells that have an endogenous TCR that recognize tumor-associated antigens, e.g. neoantigens, such as by selecting for T cells that are surface positive for one or more T cell activation marker (e.g. CD107, CD107a, CD039, CD134, CD137, CD59, CD69, CD90, CD38, or CD103).

In some embodiments, the TILs are produced by a method that includes the steps of (1) selecting, from a population of cells containing T lymphocytes obtained from a donor subject, cells positive for Chemokine (C—X—C motif) ligand 13 (CXCL13) and/or positive for an exhaustion marker from among PD-1, CD39 and/or TIGIT; and (2) stimulating the population by incubation or culture of selected cells with one or more T-cell stimulating agents of lymphocytes to produce a population of expanded T cells. In some embodiments, the methods for selection and/or stimulation are performed in a closed system. In some embodiments, only a single expansion step is carried out in the method. In some embodiments, an initial expansion (e.g. first expansion) is carried out prior to selecting the cells. In some embodiments, the provided methods further can include a secondary stimulation to further expand cells in which the further stimulation is by incubation or culture with one or more T-cell stimulating agents. In some embodiments, after the first stimulation (first expansion) and prior to the second stimulation (second expansion), the method can further include: (i) co-culturing a population of T cells in the presence of antigen presenting cells that present one or more MHC-associated non-native peptide; (4) Separating antigen presenting cells from the population of T cells in a closed system, such as by selecting for T cells containing endogenous TCR that are reactive to peptides present on the APCs, such as based on upregulation markers or activation markers on T cells following their co-culture with the APCs/peptides. In some embodiments, one or more of the steps is carried out in a closed system. In some embodiments, all of the steps is carried out in a closed system. In some embodiments, the T cell stimulating agent can include any one or more recombinant cytokines IL-2, IL-7, IL-15, IL-21, IL-25, IL-23, IL-27 or IL-25, such as generally at least IL-2 or IL-15. In some embodiments, the T cell stimulating agent can further include an anti-CD3 antibody (e.g. OKT3). In some embodiments, the T cell stimulating agents include an anti-CD3 antibody (OKT3) and/or a recombinant cytokine such as IL-2, IL-7, IL-15, IL-21, IL-25, IL-23. In some embodiments, the stimulation or any culture or incubation of the cells can be further carried out with an apoptosis inhibitor, such as Fas decoys or caspase inhibitors or any combination thereof.

In some embodiments, the TIL therapy is produced by a process that utilizes an unbiased identification and functional screening process to isolate and selectively expand the greatest breadth of tumor reactive TILs from the subject's tumor. In some embodiments, greater than 60%, greater than 70% or greater than 80% of the cells of the cell therapy are functional and potent tumor reactive T cells. In some embodiments, the TIL cell therapy is administered in an amount of least 10⁹ cells with greater than 70% functional and potent tumor reactive T cells.

In some embodiments, the subjects is pre-treated with the oncolytic virus. In some embodiments, the subject is first treated with the oncolytic virus therapy prior to isolating, enriching and expanding tumor-reactive T cells from the subject. In some embodiments, the treatment with the oncolytic virus improves or increases extraction of the tumor reactive T cells from the subject's tumor to thereby optimize the TIL harvest from the subject. In some embodiments, the provided methods including: administering the oncolytic virus to the subject, isolating or selecting T cells comprising tumor-reactive T cells from a tumor sample from the subject, selectively expanding tumor-reactive T cells, and administering the expanded tumor reactive T cells to the subject via autologous adoptive cell therapy.

According to one embodiment a method treating a subject having cancerous tumor, the method comprises delivering an oncolytic virus (OV) to the tumor the OV encodes transgenes for the generation of at least one immune enhancing compound wherein the OV infects the tumor resulting in the generation of the immune enhancing compound in a tumor micro-environment, wherein the generation of the immune enhancing compound enhances at least one of TIL trafficking to the tumor, TIL infiltration of the tumor, TIL function or TIL proliferation within the tumor.

In some embodiments, the subject receives administration of the oncolytic virus as a post-treatment after receiving the TIL cell therapy. In some embodiments, the subject is first treated with the TIL cell therapy and then is treated with the oncolytic virus. In some embodiments, subsequent treatment with the oncolytic virus optimizes or increases TIL trafficking and infiltration into solid tumors and thereby supports anti-tumor functions of infiltrating immune cells. In some embodiments, the methods include isolating or selecting T cells comprising tumor-reactive T cells from a tumor sample from the subject, selectively expanding tumor-reactive T cells, administering the expanded tumor reactive T cells to the subject via autologous adoptive cell therapy, and administering the oncolytic virus to the subject.

In some embodiments, the subject is the same subject from which the biological sample was obtained for producing the TIL cell therapy. In other embodiments the subject receiving treatment is different from the subject from which the biological sample was obtained. According to one or more embodiments, the provided method of treatment is an adoptive cell therapy with a therapeutic TIL composition containing T cells autologous to the subject, though the use of non-autologous T-cells is also contemplated in one or more embodiments.

In some embodiments, the TIL compositions provided herein are autologous to the subject to be treated. In such embodiments, the starting cells for expansion are isolated directly from a biological sample from the subject as described herein, in some cases including with enrichment for T cells positive for one or more selection marker as described and cultured under conditions for expansion as provided herein. In some aspects, the biological sample from the subject is or includes a tumor or lymph node sample and such sample tumor and an amount of such tissue is obtained, such as by resection or biopsy (e.g. core needle biopsy or fine-needle aspiration). In some embodiments, following the culturing under conditions for expansion in accord with the provided methods the cells are formulated and optionally cryopreserved for subsequent administration to the same subject for treating the cancer.

### VII. EXEMPLARY EMBODIMENTS

Among the provided embodiments are:
1. A nucleic acid comprising a recombinant Orthopoxvirus genome and one or more transgenes comprising (a) a nucleotide sequence encoding UL40 and/or (b) a nucleotide sequence encoding K5.
2. The nucleic acid of embodiment 1, wherein the recombinant Orthopoxvirus genome is derived from a Vaccinia virus genome.
3. The nucleic acid of embodiment 2, wherein the Vaccinia virus genome is derived from a genome of a Copenhagen strain of Vaccinia virus.
4. The nucleic acid of any of embodiments 1-4, wherein the Orthopoxvirus genome comprises:
(a) deletions in one or more of the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R;
(b) deletions in one or more of the following genes in the 3′ inverted terminal repeat (ITR): B21R, B22R, B23R, B24R, B25R, B26R, B27R, B28R, and B29R; and/or
(c) deletion in the B8R gene.
5. The nucleic acid of any of embodiments 1-5, wherein the Orthopoxvirus genome comprises:
(a) deletions in the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R and B20R;
(b) deletions in the following genes in the 3′ inverted terminal repeat (ITR): B21R, B22R, B23R, B24R, B25R, B26R, B27R, B28R and B29R; and
(c) deletion in the B8R gene.
6. The nucleic acid of embodiment 4 or embodiment 5, wherein the deletions in the C2L, F3L, B14R, and B29R vaccinia genes are partial deletions.
7. The nucleic acid of any of embodiments 1-6, wherein the Orthopoxvirus genome has at least 95% sequence identity to SEQ ID NO 15.
8. The nucleic acid of any of embodiments 1-6, wherein the Orthopoxvirus genome has the sequence set forth in SEQ ID NO 15.
9. The nucleic acid of any of embodiment 1-8, wherein the Orthopoxvirus genome comprises a nucleotide sequence encoding UL40.
10. The nucleic acid of any of embodiments 1-8, wherein the Orthopoxvirus genome comprises a nucleotide sequence encoding K5.
11. The nucleic acid of any of embodiments 1-8, wherein the Orthopoxvirus genome comprises (a) a nucleotide sequence encoding UL40 and (b) a nucleotide sequence encoding K5.
12. The nucleic acid of any of embodiments 1-11, wherein the nucleotide sequence encoding UL40 encodes an amino acid sequence with at least 95% sequence identity to SEQ ID NO 3.
13. The nucleic acid of any of embodiments 1-12, wherein the nucleotide sequence encoding UL40 encodes the amino acid sequence set forth in SEQ ID NO 3.
14. The nucleic acid of any of embodiments 1-13, wherein the nucleotide sequence encoding UL40 comprises a sequence with at least 95% sequence identity to SEQ ID NO 1.
15. The nucleic acid of any of embodiments 1-14, wherein the nucleotide sequence encoding UL40 comprises the sequence set forth in SEQ ID NO 1.
16. The nucleic acid of any of embodiments 1-15, wherein the nucleotide sequence encoding UL40 is operably linked to a vaccinia virus early/late promoter.
17. The nucleic acid of any of embodiments 1-16, wherein the nucleotide sequence encoding K5 encodes an amino acid sequence with at least 95% sequence identity to SEQ ID NO 6.
18. The nucleic acid of any of embodiments 1-17, wherein the nucleotide sequence encoding K5 encodes the amino acid sequence set forth in SEQ ID NO 6.
19. The nucleic acid of any of embodiments 1-18, wherein the nucleotide sequence encoding K5 comprises a sequence with at least 95% sequence identity to SEQ ID NO 2.
20. The nucleic acid of any of embodiments 1-19, wherein the nucleotide sequence encoding K5 comprises the sequence set forth in SEQ ID NO 2.
21. The nucleic acid of any of embodiments 1-20, wherein the nucleotide sequence encoding K5 is operably linked to a vaccinia virus early/late promoter.
22. The nucleic acid of any of embodiments 1-21, wherein the one or more transgenes comprises at least one further transgene comprising a nucleotide sequence encoding an immunomodulatory protein selected from the group consisting of a checkpoint inhibitor, an interleukin, a cytokine and an NK cell and/or T cell inhibitor.
23. The nucleic acid of embodiment 22, wherein the immunomodulatory protein is FMS-like tyrosine kinase 3 ligand (FLT3L), an antibody that specifically binds CTLA-4, or an Interleukin 12 (IL-12) polypeptide, optionally wherein the IL-12 polypeptide is a membrane-bound IL-12.
24. The nucleic acid of embodiment 22 or embodiment 23, wherein the nucleotide sequence encoding the at least one further transgene is operably linked to a vaccinia virus early/late promoter.
25. The nucleic acid of any of embodiments 16, 21 and 24, wherein the vaccinia virus early/late promoter is selected from H5R, P7.5, and E3L or is selected from SEQ ID NO:4, SEQ ID NO: 5, and SEQ ID NO: 14.
26. The nucleic acid of any of embodiments 1-25, wherein the one or more transgenes is inserted into locus 5p of the recombinant Orthopoxvirus genome between C2L and F3L.
27. The nucleic acid of any of embodiments 1-26, wherein the one or more transgenes are in the same orientation as endogenous Orthopoxvirus genes within the recombinant Orthopoxvirus genome.
28. The nucleic acid of any of embodiments 1-9 and 11-27, wherein expression of UL40 increases HLA-E surface expression in a host cell infected by a recombinant Orthopoxvirus comprising the nucleic acid.
29. The nucleic acid of any of embodiments 1-8 and 10-27, wherein expression of K5 decreases HLA-ABC surface expression on a host cell infected by a recombinant Orthopoxvirus comprising the nucleic acid.
30. The nucleic acid of any of embodiments 1-8 and 10-27, wherein the expression of K5 decreases ICAM-1 surface expression on a host cell infected by a recombinant Orthopoxvirus comprising the nucleic acid.
31. The nucleic acid of any of embodiments 1-30, wherein host cells infected by a recombinant Orthopoxvirus comprising the nucleic acid. are killed by lymphocytes at a reduced rate in comparison to cells infected with a reference recombinant Orthopoxvirus that has a similar genome but does not comprise a nucleotide sequence encoding either UL40 or K5.
32. The nucleic acid of any of embodiments 1-31, wherein host cells infected by a recombinant Orthopoxvirus comprising the nucleic acid are killed by NK cells at a reduced rate in comparison to cells infected with a recombinant Orthopoxvirus that has a similar genome but does not comprise a nucleotide sequence encoding either UL40 or K5.
33. The nucleic acid of any of embodiments 1-32, wherein host cells infected by a recombinant Orthopoxvirus comprising the nucleic acid are killed by T cells at a reduced rate in comparison to cells infected with a recombinant Orthopoxvirus that has a similar genome but does not comprise a nucleotide sequence encoding either UL40 or K5.
34. A recombinant Orthopoxvirus encoded by the nucleic acid of any of embodiments 1-33.
35. A pharmaceutical composition comprising the recombinant Orthopoxvirus of embodiment 34 and a physiologically acceptable carrier.
36. A method of treating cancer comprising administering the recombinant Orthopoxvirus of embodiment 34 to a subject
37. A method of treating cancer comprising administering the pharmaceutical composition of embodiment 35 to a subject
38. The method of embodiment 36 or embodiment 37, wherein the subject is human.
39. The method of any of embodiments 36-38, further comprising administering a second therapeutic agent for the treatment of cancer.
40. The method of embodiment 39, wherein the second therapeutic agent is administered before, concurrently with or after administering the pharmaceutical composition or recombinant Orthopoxvirus.
41. The method of embodiment 39 or embodiment 40, wherein the second therapeutic agent is an autologous tumor infiltrating lymphocyte (TIL) therapy.
42. The method of embodiment 41, wherein the pharmaceutical composition or recombinant Orthopoxvirus is administered to the subject prior to harvesting the TILs from a tumor from the subject for producing the autologous TIL therapy.
43. The method of embodiment 39 or embodiment 40, wherein the second therapeutic agent is CAR T cell therapy.
44. The method of embodiment 39 or embodiment 40, wherein the second therapeutic agent is a checkpoint blockade immunotherapy.
45. The method of embodiment 44, wherein the checkpoint blockade immunotherapy is a PD1 and/or a PD-L1 inhibitor.
46. The method of embodiment 39 or embodiment 40, wherein the second therapeutic agent is an immunomodulatory agent.
47. The method of embodiment 46, wherein the immunomodulatory agent is a CD47 inhibitor.
48. The method of embodiment 46, wherein the immunomodulatory agent is an NKG2A inhibitor.

### VIII. EXAMPLES

The following examples are included for illustrative purposes only and are not intended to limit the scope of the invention.

### Example 1 Generation of Oncolytic Recombinant Vaccinia Viruses Encoding Molecules to Reduce Natural Killer (NK) Cell Mediated and T Cell Mediated Antiviral Immune Responses

Recombinant vaccinia virus (VACV) was generated carrying a gene encoding a human cytomegalovirus (HCMV) glycoprotein UL40, a gene encoding Kaposi's sarcoma-associated herpesvirus (KSHV) K5 or both. UL40 and K5 were chosen as possible VACV transgene candidates to lead to expression of proteins in infected cells that could inhibit NK and T-cell mediated killing of VACV infected cells. Specifically, UL40 contains an HLA-E binding peptide and is able to promote cell surface expression of HLA-E to present the HLA-E/UL40 peptide complex to the inhibitory NK cell receptor NKG2A/CD94. K5 is a ubiquitin E3 ligase which is able to downregulate expression of MHC-I molecules (HLA-A and HLA-B) and ligands of the activating NK receptor NKG2D, and also may decrease ICAM-1 surface expression which could impair CD8⁺ T cells from binding virus infected cells.

An exemplary recombinant vaccinia virus (VACV) named Superior Killing Virus (SKV) was used as the parent virus for subsequent genetic engineering. SKV is an attenuated vector derived from the Copenhagen strain of VACV that contains 2 major deletions at the 5′ and the 3′ ends as well as the deletion of the B8R gene The SKV virus was modified by insertion of a gene encoding HCMV UL40 (UL40), a gene encoding Kaposi's sarcoma-associated herpesvirus (KSHV) K5 (K5) or both UL40 and K5.

Three recombinant VACV viruses were engineered by insertion of the open reading frame (ORF) coding for proteins UL40 (SEQ ID NO: 3; Uniprot P16780), K5 (SEQ ID NO: 6; Uniprot F5H9K4), or both into locus 5p, each under control of early/late promoters. Table E1 summarizes the recombinant viruses that were generated. In addition, GFP was inserted into the SKV to track which cells are infected. Expression of the genes were confirmed with RT-qPCR.

TABLE E1
Candidate Recombinant VACV Viruses

| Name | Transgene(s) | SEQ ID NO |
|---|---|---|
| SKV-UL40 recombinant | UL40 | 16 |
| virus | | |
| SKV-K5 recombinant virus | K5 | 17 |
| SKV-UL40_K5 recombinant | UL40 and K5 | 18 |
| virus | | |

A brief description of methods for generating recombinant viruses will now be provided. DNA homologous recombination (HR) methods for inserting exogenous DNA at genomic loci is widely used to construct Vaccinia virus (VACV) vectors. In one mor more approaches such methods may be carried out by using a shuttle vector for HR, which recombines with VACV genome in pre-infected cells.

Shuttle plasmids that contain an expression cassette together with an excisable gene marker for the rapid screening of recombinant viruses has been engineered to target the 5p locus within SKV backbone. The transgenic cassette encodes UL40 and/or K5 protein(s) for which expression is driven by two distinct early/late promoters. To note, site-specific recombination system, such as FLP-FRT, can be used to remove the gene marker (GFP) from the newly rescued virus at a later stage.

For generation of a recombinant SKV virus (expressing one or two transgene(s)), U2OS cells are infected with SKV, and then transfected with the 5p targeting shuttle plasmid. DNA recombination occurs between homologous regions in the shuttle vector and the virus, leading to the insertion of the transgene expression cassette into the targeted locus of SKV.

Recombinant SKV viruses co-expressing one or two transgene(s) and the fluorescent protein marker are isolated or purified by a standard plaque-picking procedure using fluorescence microscopy. After the last round of plaque picking, a stock of the plaque purified SKV viruses are grown in U2OS cells and used to prepare viral DNA. To confirm that our final plaque picks are the viruses of interest, the whole virus genome is analyzed by next generation sequencing. A purified population of a clone with no mutation will move a step forward in the characterization process.

### Example 2 Assessment of UL40 and K5 Activity and NK Cell Mediated Killing of Virus Infected Cells

The recombinant viruses described in Example 1 were used to determine if UL40 and K5 retain their activity in the context of VACV infection and to assess their effect on NK cell mediated killing of recombinant virus infected cells. Recombinant virus infected cells were compared to the parental SKV infected cells.

### A. Assessment of K5 and UL40 Activity in Infected HeLa Cells

In the studies, HeLa cells were used as host cells to test for NK killing. To assess if activity of UL40 and K5 is retained in infected HeLa cells, confluent and adherent HeLa cells were infected with recombinant viruses or parental SKV at an MOI of 0.1 and incubated for 18-20 hours. Infected HeLa cells were stained with antibodies directed against ICAM-I, HLA-ABC, and HLA-E to assess their surface expression. Mean fluorescence intensity (MFI) and frequency of cells positive for the surface markers were measured on virus infected cells expressing GFP by flow cytometry.

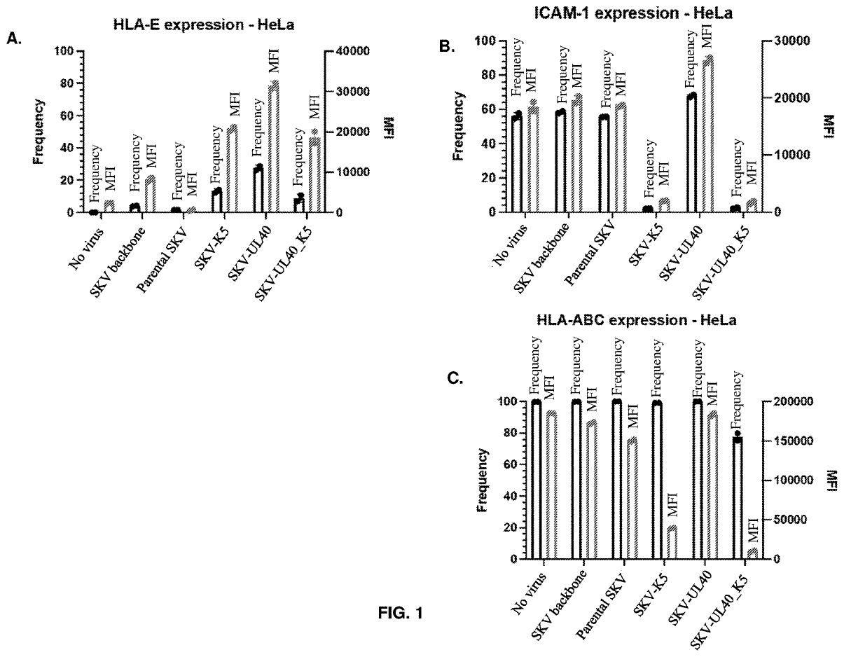

Hela cells infected with SKV-UL40 recombinant virus had increased HLA-E surface expression, with a 1.5-fold increase in HLA-E expression compared to HeLa cells infected with SKV-K5 recombinant virus and a 3-fold increase in expression compared to the parental SKV virus (FIG. 1A).

As shown in FIG. 1B, HeLa cells infected with SKV-K5 recombinant virus exhibited a drastic decrease in ICAM-1 surface expression, with approximately a 20-fold decrease in ICAM-I surface expression as determined by flow cytometry compared to the parental SKV virus infected HeLa cells.

As shown in FIG. 1C, HeLa cells infected with K5 recombinant virus also had decreased surface expression of HLA-ABC with approximately a 3-fold decrease in MFI compared to HeLa cells infected with the parental SKV virus. As expected, UL40 delivery by SKV did not impact HLA-ABC surface expression (FIG. 1C). Further, the increase in HLA-E expression shown in FIG. 1A in HeLa cells infected with SKV-K5 recombinant virus is consistent with the activity of K5 to indirectly increase HLA-E surface expression by promoting endocytosis of HLA-ABC molecules but not HLA-E.

Characterization of HeLa cells infected with SKV expressing both UL40 and K5 showed an impact on all assessed markers. HLA-E expression was increased by approximately 2-fold in HeLa cells infected with SKV-UL40_K5 recombinant virus compared to the parental SKV virus infected cells, as shown in FIG. 1A. ICAM-1 surface expression was decreased by approximately 20-fold in SKV-UL40_K5 recombinant virus infected cells compared to the parental SKV virus infected cells, as shown in FIG. 1B. HLA-ABC surface expression also was downregulated by approximately 10-fold SKV-UL40_K5 recombinant virus infected cells compared to parental SKV virus infected cells, as shown in FIG. 1C.

Together, these data clearly demonstrate that delivery of UL40 and K5, individually or together, retain their activity in the context of VACV infection. HLA-E surface expression is upregulated in cells infected with UL40 expressing VACV while MHCI molecules (HLA-ABC) and ICAM-1 are downregulated from the surface in cells infected with K5 expressing VACV. In addition, fitness analysis of UL40(+/−K5) VACV revealed no difference in comparison with VACV (data not shown).

### B. NK Cell Killing Assay

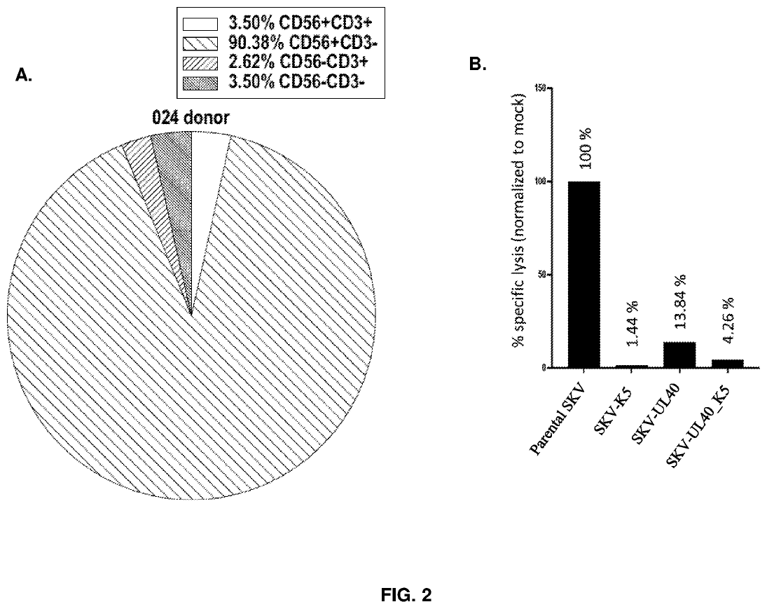

The infected HeLa cells described in the above experiments were used as target cells to assess NK cell mediated killing. The HeLa cells were labeled with cell trace violet to monitor cell proliferation and infected with either SKV, SKV-UL40, SKV-K5 or SKV-UL40_K5 as described above. All SKV viruses also expressed GFP to monitor infection. To obtain NK cells, NK cells were expanded from human peripheral blood mononuclear cells (PBMCs) for 14 days using the CellXVivo Human NK cell expansion kit (R&D Systems), then purified using the NK cell enrichment kit from Miltenyi Biotec. Purity of isolated CD56+/CD3− NK cells was assessed by flow cytometry as depicted in FIG. 2A.

The enriched NK cells were co-cultured with approximately 50,000 SKV-infected HeLa cells per well of a 96 well plate at a 5:1 ratio of effector cells to target cells and incubated at 37° C. for about 5 hours. The cells were then stained for viability and NK cell cytotoxicity and analyzed by flow cytometry.

Results indicated that NK-mediated cytotoxicity was drastically reduced in HeLa cells infected with recombinant SKV expressing UL40 and/or K5. As depicted in FIG. 2B, there was reduced NK cell killing of SKV-UL40 recombinant virus infected cells (86.16% reduction), SKV-K5 recombinant virus infected cells (98.56% reduction), and SKV-UL40_K5 recombinant virus infected cells (95.72% reduction) compared to the parental SKV infected cells. These results demonstrate that all three recombinant viruses are protected from NK cell killing and both UL40 and K5 maintain their NK cell inhibitory effect in a recombinant vaccinia virus.

### Example 3 Assessment of UL40 and K5 on CD8⁺ T Cell Mediated Killing of Oncolytic Virus Infected Cells

The recombinant viruses described in Example 1 were used to assess if UL40 and/or K5 expression by VACV can protect the infected cells from killing mediated by CD8+ T cells.

### A. Assessment of K5 and UL40 Activity in Infected MHCI Antigen-Expressing Target Cells

MeWo_MART-1 cell lines were used as target cells for infection with recombinant viruses and parental SKV for use in T cell killing assay. The MeWo cell line expresses the melanoma tumor-associated antigen MART-1 that will be presented by MHCI at the cell surface for recognition by specific MART-1-specific CD8+ T cells. MeWo cells were seeded at 40,000 cells per well (96-well plate), infected with recombinant SKV viruses (MOI 0.1), stimulated with recombinant human IFNγ (100 ng/mL), and incubated at 37° C. for 18-21 hours. A pool of virus infected MeWo_MART-1 target cells were stained with antibodies targeting HLA-ABC and HLA-E and mean fluorescence intensity (MFI) was measured on virus infected MeWo_MART-1 target cells expressing GFP using flow cytometry.

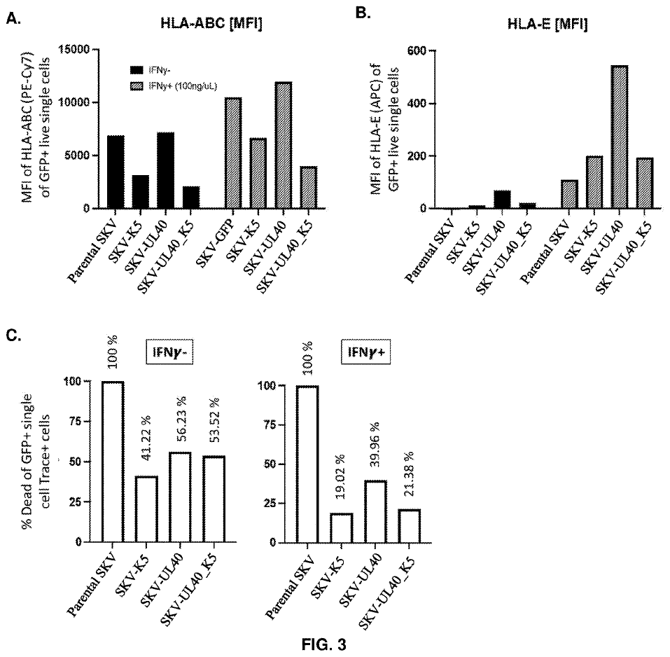

As shown in FIG. 3A, HLA-ABC surface expression is upregulated in the presence of IFNγ compared to cultures incubated without IFNγ. In both IFNγ⁺ and IFNγ⁻ cultures, the SKV-UL40 recombinant virus had no effect on HLA-ABC surface expression compared to the parental SKV on infected cells. As displayed in FIG. 3B, MeWo_MAR-1 cells infected with SKV-UL40 recombinant virus had increased expression of HLA-E in both IFNγ positive and negative cultures compared to cells infected with the parental SKV.

MeWo_MART-1 cells infected with the K5 recombinant virus had decreased HLA-ABC surface expression by up to 3-fold in cultures with and without IFNγ when compared to cells infected with the parental SKV, as shown in FIG. 3A. HLA-E expression was unaffected or moderately increased in the K5 recombinant virus infected cells compared to the parental SKV infected cells, as shown in FIG. 3B.

MeWo_MART-1 cells infected with SKV-UL40_K5 recombinant virus had decreased expression of HLA-ABC in both IFNγ⁺ and IFNγ⁻ cultures compared to cells infected with the parental SKV, as shown in FIG. 3A. SKV-UL40-K5 recombinant virus infected cells also had modest increases in HLA-E surface expression compared to cells infected with the parental SKV, as shown in FIG. 3B.

### B. CD8⁺ T Cell Killing Assay

The infected MeWo_MART-1 cells described in the above experiments were used as target cells to assess T cell mediated killing. The T cell killing assay was performed using MART-1-specific CD8+ T-cells incubated with MeWo_MART-1 cells infected with either parental SKV, SKV-UL40 recombinant virus, SKV-K5 recombinant virus, or SKV-UL40_K5 recombinant virus in the presence or absence of IFNγ as described above. All SKV viruses also expressed GFP to monitor infection. Effector T cells were incubated for 18-21 hours (overnight) in 300 IU/mL of recombinant human IL-2 before being added to infected target cells at a ratio of 3:1 effector cells to target cells. The co-culture was incubated at 37° C. while maintaining the 300 IU/mL concentration of recombinant human IL-2. After 6 hours of incubation, the co-culture was then stained for cell viability and analyzed by flow cytometry.

As displayed in FIG. 3C, in the absence of IFNγ, T cell mediated cytotoxicity was reduced in MeWo_MART-1 cells infected with the SKV-UL40 recombinant virus by 43.77% compared to the parental SKV infected cells. In the absence of IFNγ, T cell mediated cytotoxicity was also reduced in SKV-K5 recombinant virus infected cells and SKV-UL40_K5 recombinant virus infected cells by 58.78% and 46.48% respectively when compared to cells infected with parental SKV. The presence of IFNγ markedly increased T cell-mediated killing, likely by upregulating MHCI expression on the target cells. In the presence of IFNγ, CD8⁺ T cell mediated cytotoxicity was reduced in SKV-UL40 recombinant virus infected cells, SKV-K5 recombinant virus infected cells, and SKV-UL40_K5 recombinant virus infected cells by 60.04%, 80.98%, and 78.62% respectively when compared to the parental SKV infected cells.

Inhibition of CD8⁺ T cell mediated cell lysis by UL40 expressed in infected cells was unexpected, and is believed to be the first time UL40 has been demonstrated to protect virus infected cells against killing mediated by T cells. Without wishing to be bound by theory, the inhibition of T cell mediated killing by delivering UL40 to infected cells is likely due to the UL40 recombinant virus upregulation of HLA-E. It is likely that HLA-E/UL40 peptide complexes with the inhibitory receptor NKG2A/CD94 expressed on activated CD8⁺ T cells to decrease their cytotoxic activity.

Together with the results in Example 2, these data demonstrate that UL40 and/or K5 protein(s) expressed by VACV protect(s) infected cells from killing mediated by both NK and T cells. Importantly, K5 and UL40 inhibit killing mediated by NK and T cells through different mechanisms which would be expected to expand their range of action when used together. This is of particular importance in oncolytic virotherapy while targeting heterogenous tumour cells that can, in some cases, be both positive and negative for MHCI expression. Further, K5 and UL40 delivered by VACV are retained within the cell interior which restricts their protective function to the infected cells. Such recombinant VACV are suitable to be used in combination with adoptive T cell therapy, such as using autologous bulk tumour-infiltrating lymphocytes (TIL) or enriched or selected tumor reactive TILs. Recombinant VACV can be administrated prior to, concurrently with or after treatment with TIL transfer.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

### CONCLUSION

The foregoing descriptions of various embodiments of the invention have been presented for purposes of illustration and description. It is not intended to limit the invention to the precise forms disclosed. Many modifications, variations and refinements will be apparent to practitioners skilled in the art. For example, embodiments of the combination of oncolytic virus and TIL therapy can be adapted for a number of cancers including solid tumors such as GI, breast, bone, colorectal, and melanomas and various liquid tumors such as leukemia. Also, those skilled in the art will recognize, or be able to ascertain using no more than routine experimentation, numerous equivalents to the specific devices and methods described herein. Such equivalents are considered to be within the scope of the present invention and are covered by the appended claims below.

Elements, characteristics, or acts from one embodiment can be readily recombined or substituted with one or more elements, characteristics or acts from other embodiments to form numerous additional embodiments within the scope of the invention. Moreover, elements that are shown or described as being combined with other elements, can, in various embodiments, exist as standalone elements. Further still, embodiments of the invention also contemplate the exclusion or negative recitation of an element, feature, chemical, therapeutic agent, characteristic, value or step wherever said element, feature, chemical, therapeutic agent, characteristic, value, step or the like is positively recited. Hence, the scope of the present invention is not limited to the specifics of the described embodiments, but is instead limited solely by the appended claims.

What is claimed is:

1. A nucleic acid comprising a recombinant Orthopoxvirus genome and one or more transgenes comprising (a) a nucleotide sequence encoding UL40 and/or (b) a nucleotide sequence encoding K5.

2. The nucleic acid of claim 1, wherein the recombinant Orthopoxvirus genome is derived from a Vaccinia virus genome.

3. The nucleic acid of claim 2, wherein the Vaccinia virus genome is derived from a genome of a Copenhagen strain of Vaccinia virus.

4. The nucleic acid of any of claims 1-3, wherein the Orthopoxvirus genome comprises:
(a) deletions in one or more of the following genes: C2L, C1L, N1L, N2L, MIL, M2L, K1L, K2L, K3L, K4L, K5L, K6L, K7R, F1L, F2L, F3L, B14R, B15R, B16R, B17L, B18R, B19R, and B20R;
(b) deletions in one or more of the following genes in the 3′ inverted terminal repeat (ITR): B21R, B22R, B23R, B24R, B25R, B26R, B27R, B28R, and B29R; and/or
(c) deletion in the B8R gene.

5. The nucleic acid of any of claims 1-4, wherein the Orthopoxvirus genome comprises:
(a) deletions in the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R and B20R;
(b) deletions in the following genes in the 3′ inverted terminal repeat (ITR): B21R, B22R, B23R, B24R, B25R, B26R, B27R, B28R and B29R; and
(c) deletion in the B8R gene.

6. The nucleic acid of claim 4 or claim 5, wherein the deletions in the C2L, F3L, B14R, and B29R vaccinia genes are partial deletions.

7. The nucleic acid of any of claims 1-6, wherein the Orthopoxvirus genome has at least 95% sequence identity to SEQ ID NO 15.

8. The nucleic acid of any of claims 1-6, wherein the Orthopoxvirus genome has the sequence set forth in SEQ ID NO 15.

9. The nucleic acid of any of claim 1-8, wherein the Orthopoxvirus genome comprises a nucleotide sequence encoding UL40.

10. The nucleic acid of any of claims 1-8, wherein the Orthopoxvirus genome comprises a nucleotide sequence encoding K5.

11. The nucleic acid of any of claims 1-8, wherein the Orthopoxvirus genome comprises (a) a nucleotide sequence encoding UL40 and (b) a nucleotide sequence encoding K5.

12. The nucleic acid of any of claims 1-11, wherein the nucleotide sequence encoding UL40 encodes an amino acid sequence with at least 95% sequence identity to SEQ ID NO 3.

13. The nucleic acid of any of claims 1-12, wherein the nucleotide sequence encoding UL40 encodes the amino acid sequence set forth in SEQ ID NO 3.

14. The nucleic acid of any of claims 1-13, wherein the nucleotide sequence encoding UL40 comprises a sequence with at least 95% sequence identity to SEQ ID NO 1.

15. The nucleic acid of any of claims 1-14, wherein the nucleotide sequence encoding UL40 comprises the sequence set forth in SEQ ID NO 1.

16. The nucleic acid of any of claims 1-15, wherein the nucleotide sequence encoding UL40 is operably linked to a vaccinia virus early/late promoter.

17. The nucleic acid of any of claims 1-16, wherein the nucleotide sequence encoding K5 encodes an amino acid sequence with at least 95% sequence identity to SEQ ID NO 6.

18. The nucleic acid of any of claims 1-17, wherein the nucleotide sequence encoding K5 encodes the amino acid sequence set forth in SEQ ID NO 6.

19. The nucleic acid of any of claims 1-18, wherein the nucleotide sequence encoding K5 comprises a sequence with at least 95% sequence identity to SEQ ID NO 2.

20. The nucleic acid of any of claims 1-19, wherein the nucleotide sequence encoding K5 comprises the sequence set forth in SEQ ID NO 2.

21. The nucleic acid of any of claims 1-20, wherein the nucleotide sequence encoding K5 is operably linked to a vaccinia virus early/late promoter.

22. The nucleic acid of any of claims 1-21, wherein the one or more transgenes comprises at least one further transgene comprising a nucleotide sequence encoding an immunomodulatory protein selected from the group consisting of a checkpoint inhibitor, an interleukin, a cytokine and an NK cell and/or T cell inhibitor.

23. The nucleic acid of claim 22, wherein the immunomodulatory protein is FMS-like tyrosine kinase 3 ligand (FLT3L), an antibody that specifically binds CTLA-4, or an Interleukin 12 (IL-12) polypeptide, optionally wherein the IL-12 polypeptide is a membrane-bound IL-12.

24. The nucleic acid of claim 22 or claim 23, wherein the nucleotide sequence encoding the at least one further transgene is operably linked to a vaccinia virus early/late promoter.

25. The nucleic acid of any of claims 16, 21 and 24, wherein the vaccinia virus early/late promoter is selected from H5R, P7.5, and E3L or is selected from SEQ ID NO:4, SEQ ID NO: 5, and SEQ ID NO: 14.

26. The nucleic acid of any of claims 1-25, wherein the one or more transgenes is inserted into locus 5p of the recombinant Orthopoxvirus genome between C2L and F3L.

27. The nucleic acid of any of claims 1-26, wherein the one or more transgenes are in the same orientation as endogenous Orthopoxvirus genes within the recombinant Orthopoxvirus genome.

28. The nucleic acid of any of claims 1-9 and 11-27, wherein expression of UL40 increases HLA-E surface expression in a host cell infected by a recombinant Orthopoxvirus comprising the nucleic acid.

29. The nucleic acid of any of claims 1-8 and 10-27, wherein expression of K5 decreases HLA-ABC surface expression on a host cell infected by a recombinant Orthopoxvirus comprising the nucleic acid.

30. The nucleic acid of any of claims 1-8 and 10-27, wherein the expression of K5 decreases ICAM-1 surface expression on a host cell infected by a recombinant Orthopoxvirus comprising the nucleic acid.

31. The nucleic acid of any of claims 1-30, wherein host cells infected by a recombinant Orthopoxvirus comprising the nucleic acid. are killed by lymphocytes at a reduced rate in comparison to cells infected with a reference recombinant Orthopoxvirus that has a similar genome but does not comprise a nucleotide sequence encoding either UL40 or K5.

32. The nucleic acid of any of claims 1-31, wherein host cells infected by a recombinant Orthopoxvirus comprising the nucleic acid are killed by NK cells at a reduced rate in comparison to cells infected with a recombinant Orthopoxvirus that has a similar genome but does not comprise a nucleotide sequence encoding either UL40 or K5.

33. The nucleic acid of any of claims 1-32, wherein host cells infected by a recombinant Orthopoxvirus comprising the nucleic acid are killed by T cells at a reduced rate in comparison to cells infected with a recombinant Orthopoxvirus that has a similar genome but does not comprise a nucleotide sequence encoding either UL40 or K5.

34. A recombinant Orthopoxvirus encoded by the nucleic acid of any of claims 1-33.

35. A pharmaceutical composition comprising the recombinant Orthopoxvirus of claim 34 and a physiologically acceptable carrier.

36. A method of treating cancer comprising administering the recombinant Orthopoxvirus of claim 34 to a subject

37. A method of treating cancer comprising administering the pharmaceutical composition of claim 35 to a subject

38. The method of claim 36 or claim 37, wherein the subject is human.

39. The method of any of claims 36-38, further comprising administering a second therapeutic agent for the treatment of cancer.

40. The method of claim 39, wherein the second therapeutic agent is administered before, concurrently with or after administering the pharmaceutical composition or recombinant Orthopoxvirus.

41. The method of claim 39 or claim 40, wherein the second therapeutic agent is an autologous tumor infiltrating lymphocyte (TIL) therapy.

42. The method of claim 41, wherein the pharmaceutical composition or recombinant Orthopoxvirus is administered to the subject prior to harvesting the TILs from a tumor from the subject for producing the autologous TIL therapy.

43. The method of claim 39 or claim 40, wherein the second therapeutic agent is CAR T cell therapy.

44. The method of claim 39 or claim 40, wherein the second therapeutic agent is a checkpoint blockade immunotherapy.

45. The method of claim 44, wherein the checkpoint blockade immunotherapy is a PD1 and/or a PD-L1 inhibitor.

46. The method of claim 39 or claim 40, wherein the second therapeutic agent is an immunomodulatory agent.

47. The method of claim 46, wherein the immunomodulatory agent is a CD47 inhibitor.

48. The method of claim 46, wherein the immunomodulatory agent is an NKG2A inhibitor.
